(19) United States
(12) Patent Application Publication
(10) Pub. No.: US 2024/0199588 A1
(43) Pub. Date: Jun. 20, 2024

(54) Chiral TACN/NOTA compounds/derivatives with and without metals for application

(71) Applicant: The Hong Kong Polytechnic University, Hong Kong (CN)

(72) Inventor: Ga-Lai LAW, Hong Kong (CN)

(21) Appl. No.: 18/550,178

(22) PCT Filed: Mar. 21, 2022
(86) PCT No.: PCT/CN2022/081959

§ 371 Date: Sep. 12, 2023

Related U.S. Application Data
(60) Provisional application No. 63/163,105, filed on Mar. 19, 2021.

(51) Int. Cl. C07D 403/14; A61K 49/00; A61K 49/10; A61K 51/04; C07D 255/02; C07D 401/14

(52) U.S. Cl. CPC C07D 403/14; A61K 49/0002; A61K 49/106; A61K 51/0482; C07D 255/02; C07D 401/14

(57) ABSTRACT

Cyclic 1,4,7-triazacyclononane-1,4,7-triacetic acid chelators and metal complexes comprising the same useful as positron emission tomography imaging agents, magnetic resonance imaging contrast agents, and computed tomography imaging agents, and optical imaging agents, and methods of use and preparation thereof.

### CROSS-REFERENCE TO RELATED APPLICATIONS

The present application claims priority from U.S. Provisional Patent Application No. 63/163,105, filed on Mar. 19, 2021, which is hereby incorporated by reference in its entirety.

### TECHNICAL FIELD

The present disclosure generally relates to positron emission tomography (PET) agents, magnetic resonance imaging (MRI) contrast agents, computed tomography (CT) imaging agents, and optical imaging and methods of use and preparation thereof.

### BACKGROUND

Cyclic 1,4,7-triazacyclononane-1,4,7-triacetic acid (NOTA) chelator and its derivatives have many applications. They are widely used as radiometal chelators for PET imaging, lanthanide chelators for MRI contrast agents, electron paramagnetic resonance (EPR) tags as well as luminescent materials for optical imaging applications. It has reported that introducing chiral substituents onto an achiral chelator can make complexes therefrom more rigid and can improve stability. The reduced number of stable conformations also make the formed complex promising as nuclear magnetic resonance (NMR) tags for proteins and for photoluminescence (PL)/circularly polarized luminescence (CPL) as well as for MRI applications.

There thus exists a need for improved chiral NOTA chelators and complexes that exhibit at least some of the improved properties described above.

### SUMMARY

In a first aspect, provided herein is a chiral NOTA chelator of Formula 1:

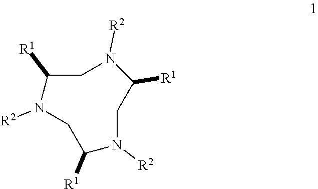

or a pharmaceutically acceptable salt or zwitterion thereof, wherein
R¹ is selected from the group consisting of hydrogen, alkyl, alkene, alkyne, cycloalkyl, heterocycloalkyl, aryl, heteroaryl, araalkyl, and —(CR₂)ₙY, wherein n is a whole number selected from 1-10; each R is independently hydrogen, alkyl, cycloalkyl, or aryl; or two R taken together with the carbon(s) to which they are attached form a 3-6 membered cycloalkyl; and Y is hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R¹ is a side chain of a naturally occurring amino acid or the side chain of a D-isomer of a naturally occurring amino acid; or R¹ is a moiety having the structure:

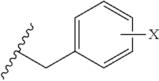

wherein X is azide, alkyne, halide, tosylate, mesylate, or hydroxyl; and
R² is —(C═O)OH, —(C═O)NHR⁵, or —(CH₂)ₘZ, wherein m is a whole number selected from 2-8; R⁵ is a targeting agent; and Z is moiety of Formula 2:

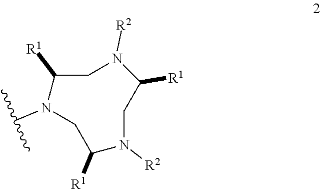

or a pharmaceutically acceptable salt or zwitterion thereof; or R² is a moiety of Formula 3:

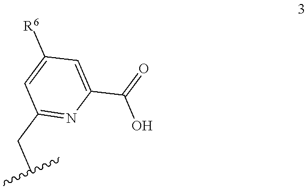

or a pharmaceutically acceptable salt or zwitterion thereof, wherein R⁶ for each occurrence is independently hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R⁶ is a moiety of Formula 4:

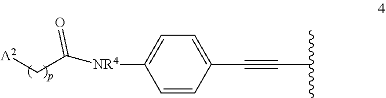

wherein p is a whole number selected from 1-6;
each A² is independently —CO₂R⁵, —NHR⁵, —OR⁵, N₃, or alkyne; and R⁴ is hydrogen or alkyl, with the proviso that if one R² is —(CH₂)₂Z and four R² are each —(C═O)OH, then each R¹ cannot be hydrogen; and if three R² are each —(C═O)OH, then each R¹ cannot be hydrogen.

In certain embodiments, each R¹ is selected from the group consisting of hydrogen, alkyl, aryl, heteroaryl, and —(CR₂)ₙY, wherein Y is heteroaryl or aryl; and n is 1-4.

In certain embodiments, each R² is —(C═O)OH; or each R² is —(C═O)NHR⁵.

In certain embodiments, the chiral NOTA chelator has Formula 5:

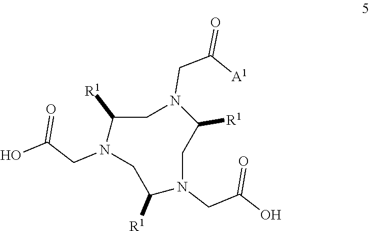

or a pharmaceutically acceptable salt or zwitterion thereof, wherein
A¹ is OH or NHR⁵;
each R¹ is selected from the group consisting of hydrogen, alkyl, aryl, heteroaryl, and —(CR₂)ₙY, wherein Y is heteroaryl or aryl; and n is 1-4; and
R⁵ is a targeting agent.

In certain embodiments, each R¹ is C₁-C₆ alkyl; or each R¹ is —(CR₂)ₙY, wherein n is a whole number selected from 1-4; and Y is aryl or heteroaryl.

In certain embodiments, each R¹ is ethyl; or each R¹ is 3-(λ³-methyl)-1H-indole,

In certain embodiments, the chiral NOTA chelator has Formula 7 or Formula 8

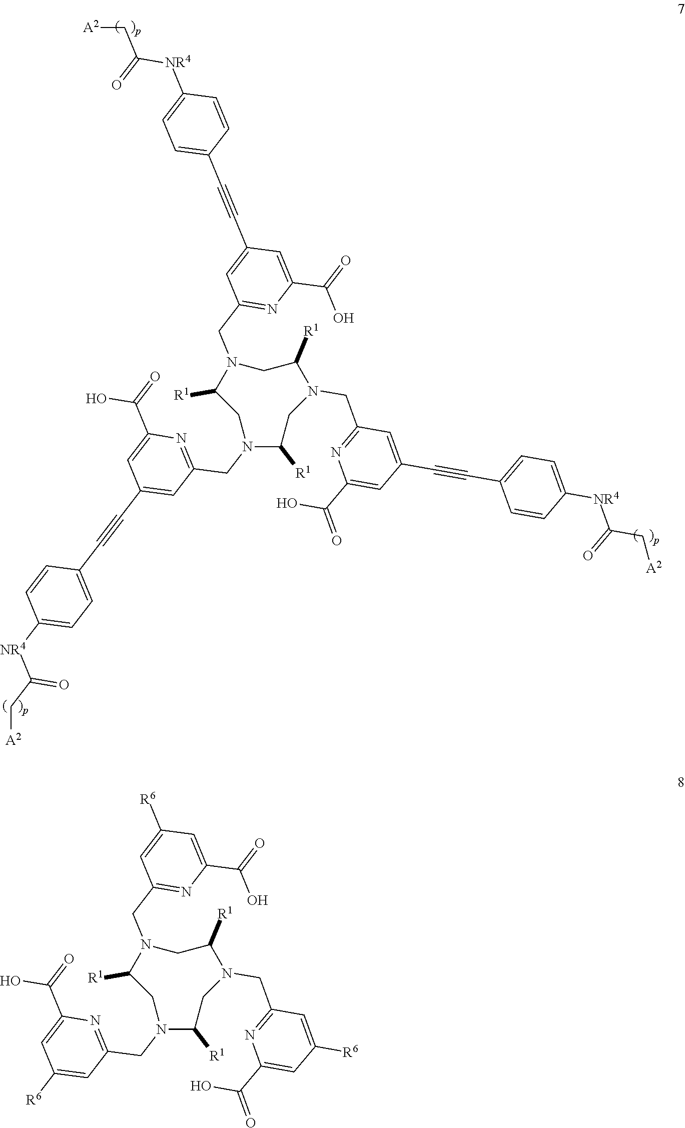

or a pharmaceutically acceptable salt or zwitterion thereof, wherein
p is a whole number selected from 1-4;
each A² is independently —CO₂R⁵, —NHR⁵, —OR⁵, N₃, or alkyne;
R¹ is selected from the group consisting of hydrogen, alkyl, aryl, heteroaryl, and —(CR₂)ₙY, wherein Y is heteroaryl or aryl; and n is 1-4;
R⁴ is hydrogen or alkyl;
R⁵ is hydrogen or a targeting agent; and
R⁶ for each occurrence is independently hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl.

In certain embodiments, wherein each R¹ is C₁-C₆ alkyl; and R⁶ for each occurrence is independently hydrogen, alkyne, halide, —N₃, —R⁵, —NH₂, or —(C═O)OH.

In certain embodiments, p is a whole number selected from 1-2; each A² is independently —CO₂R⁵; each R¹ is C₁-C₆ alkyl; R⁴ is hydrogen; and R⁶ is hydrogen.

In certain embodiments, R¹ is ethyl; and R⁵ is hydrogen.

In certain embodiments, the chiral NOTA chelator has Formula 6:

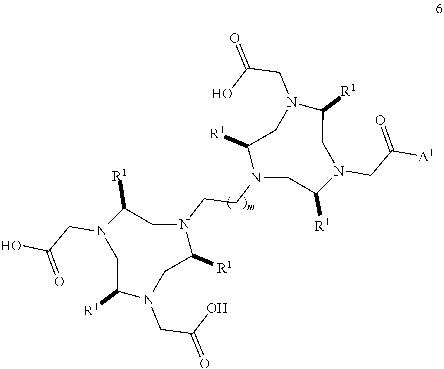

or a pharmaceutically acceptable salt or zwitterion thereof, wherein
m is a whole number selected from 2-8;
A¹ is OH or NHR⁵;
R¹ is selected from the group consisting of hydrogen, alkyl, alkene, alkyne, cycloalkyl, heterocycloalkyl, aryl, heteroaryl, araalkyl, and —(CR₂)ₙY, wherein n is a whole number selected from 1-10; each R is independently hydrogen, alkyl, cycloalkyl, or aryl; or two R² taken together with the carbon(s) to which they are attached form a 3-6 membered cycloalkyl; and Y is hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R¹ is a side chain of a naturally occurring amino acid or the side chain of a D-isomer of a naturally occurring amino acid; or R¹ is a moiety having the structure:

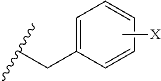

wherein X is azide, alkyne, halide, tosylate, mesylate, or hydroxyl; and
R⁵ is a targeting agent.

In certain embodiments, each R¹ is C₁-C₆ alkyl; and m is a whole number selected from 2-4.

In certain embodiments, R¹ is ethyl.

In certain embodiments, the chiral NOTA chelator is selected from the group consisting of:

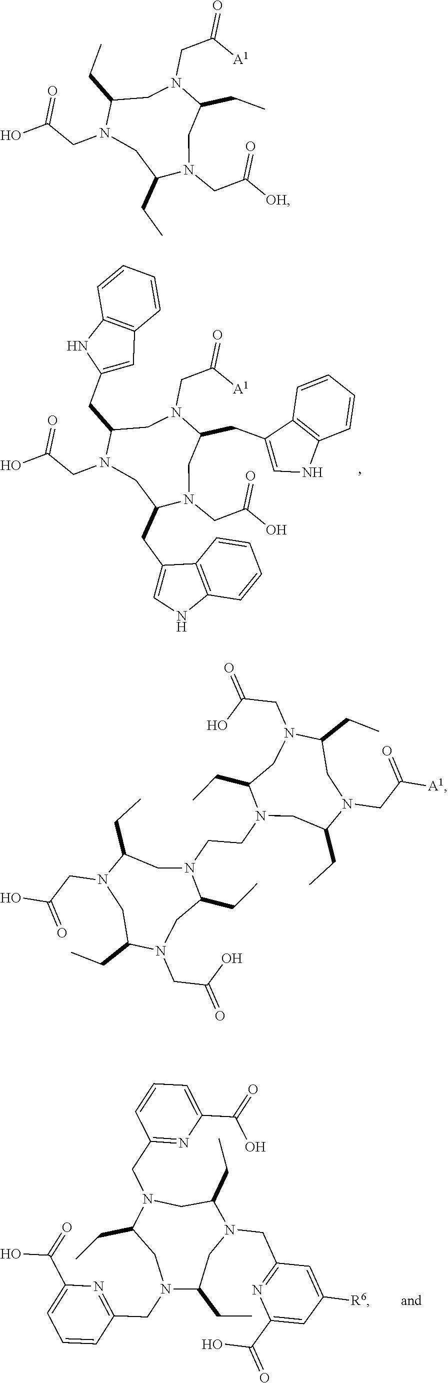

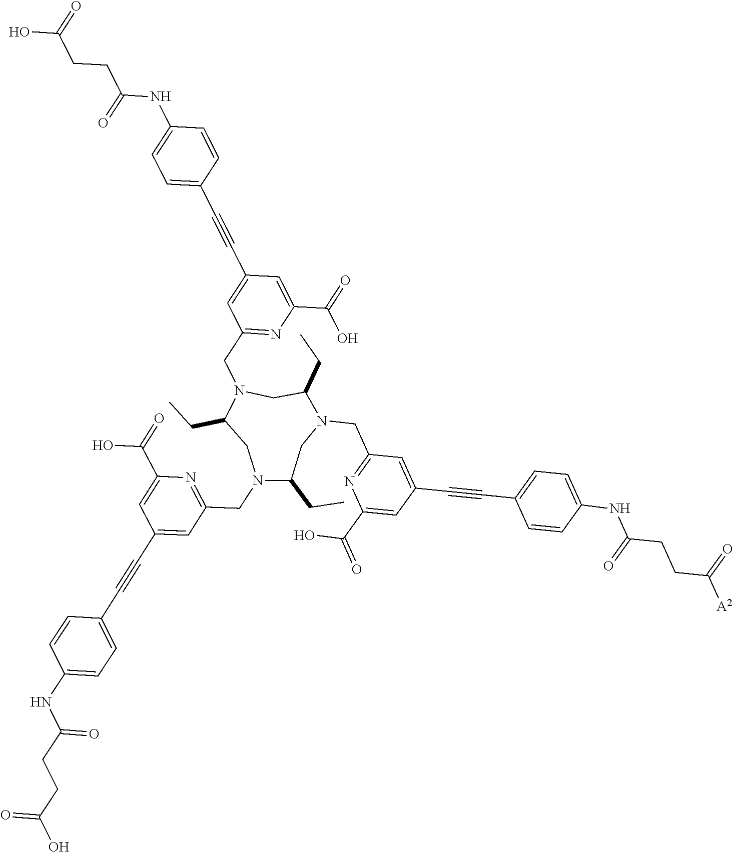

or a pharmaceutically acceptable salt or zwitterion thereof, wherein A¹ is OH or NHR⁵;
A² is OH or NHR⁵; and R⁶ is hydrogen or R⁵.

In a second aspect, provided herein is a chiral NOTA complex comprising a chiral NOTA chelator described herein and at least one metal.

In certain embodiments, the at least one metal is a Group 8-13 element of the periodic table, a lanthanide, or an actinide.

In certain embodiments, the at least one metal is Gd, Eu, Tb, Lu, Yb, Y, In, or Mn.

In a third aspect, provided herein is a pharmaceutical composition comprising a chiral NOTA complex described herein and at least one pharmaceutically acceptable excipient.

In a fourth aspect, provided herein is a chiral NOTA complex described herein for use in imaging a sample.

In certain embodiments, the imaging comprises positron emission tomography (PET), magnetic resonance imaging (MRI), computed tomography (CT) imaging, or optical imaging.

In a fifth aspect, provided herein is a chiral NOTA complex described herein for use in imaging a subject.

In certain embodiments, the imaging comprises positron PET, MRI, CT, or optical imaging.

### BRIEF DESCRIPTION OF THE DRAWINGS

The above and other objects and features of the present disclosure will become apparent from the following description of the disclosure, when taken in conjunction with the accompanying drawings.

FIG. 1 depicts the high resolution mass spectroscopy (HRMS) spectra of the purified Et-NOTA, m/z (ESI-HRMS+) 388.2449 ([M+H]+ calculated: 388.2488).

FIG. 2 depicts the HRMS of purified Mn-Et-NOTA, m/z (ESI-HRMS⁺) 441.1673 ([M+2H]⁺ calculated: 441.1672).

FIG. 3 depicts the low-resolution mass spectrum of Et-ENOTA. FIG. 4 depicts the low-resolution mass spectrum of Mn-Et-ENOTA.

FIG. 5 depicts the ¹H-NMR spectra of Eu-Py1-Et-NOTA.

FIG. 6 depicts the low-resolution mass spectrum of Py2-Et-NOTA.

FIG. 7 depicts the ¹H-NMR spectra Et-NOTA.

FIG. 8 depicts the ¹³C-NMR spectra of Et-NOTA.

FIG. 9 depicts the ¹H-NMR spectra of Et-ENOTA.

FIG. 10 depicts the ¹³C-NMR spectra of Et-NOTA.

FIG. 11 depicts the ¹H-NMR spectra of comparative achiral ENOTA.

FIG. 12 depicts the ¹³C-NMR spectra of comparative achiral ENOTA.

FIG. 13 depicts the ¹H-NMR spectra of Et-ENOTA.

FIG. 14 depicts the ¹³C-NMR spectra of Et-ENOTA.

FIG. 15 depicts the ¹H-NMR spectra of Py1-Et-NOTA.

FIG. 16 depicts the ¹³C-NMR spectra of Py1-Et-NOTA.

FIG. 17 depicts the ¹H-NMR spectra of Py2-Et-NOTA.

FIG. 18 depicts the ¹³C-NMR spectra of Py2-Et-NOTA.

FIG. 19 depicts a table showing the relaxivity of Mn-Et-NOTA.

FIG. 20 depicts a graph showing the r₁ of Mn-Et-NOTA in water as a function of concentration.

FIG. 21 depicts a graph showing the r₂ of Mn-Et-NOTA in 4.5% human serum albumin (HSA) in water as a function of concentration.

FIG. 22 depicts a graph showing the r₁ of Mn-Et-NOTA in 4.5% HSA in water as a function of concentration.

FIG. 23 depicts a graph showing the r₂ of Mn-Et-NOTA in 4.5% HSA in water as a function of concentration.

FIG. 24 depicts circularly polarized photoluminescence (CPL) emission; photoluminescence (PL) emission; and luminescence dissymmetry factor (gₗᵤₘ) of Eu-Py1-Et-NOTA, 5% DMSO in 0.1M HEPES. slit=15-7 nm, Ex=272 nm, Cycles=5.

FIG. 25 depicts CPL emission; PL emission; and gₗᵤₘ of Tb-Py1-Et-NOTA, 5% DMSO in 0.1M HEPES. slit=12-3 nm, Ex=272 nm, Cycles=5.

FIG. 26 depicts CPL emission; PL emission; and gₗᵤₘ of Eu-Py2-Et-NOTA, 5% DMSO in 0.1M HEPES. slit=15-4 nm, Ex=337 nm, Cycles=5.

FIG. 27 depicts CPL emission; PL emission; and gₗᵤₘ of Eu-Py2-Et-NOTA, 5% DMSO in 0.1M HEPES, in the presence of 1.4 T magnet (S TO N). slit=15-4 nm, Ex=337 nm, Cycles=5.

FIG. 28 depicts CPL emission; PL emission; and gₗᵤₘ of Eu-Py2-Et-NOTA, 5% DMSO in 0.1M HEPES, in the presence of 1.4 T magnet (N TO S). slit=15-4 nm, Ex=337 nm, Cycles=5.

FIG. 29 depicts a comparison of the gₗᵤₘ of Eu-Py2-Et-NOTA with and without 1.4 T magnet & different directions.

FIG. 30 depicts a comparison of the CPL of Eu-Py2-Et-NOTA with and without 1.4 T magnet & different directions.

FIG. 31 depicts a comparison of the CPL of Eu-Py2-Et-NOTA with 1.4 T magnet in different directions.

FIG. 32 depicts a comparison of the PL of Eu-Py2-Et-NOTA with and without 1.4 T magnet in different directions.

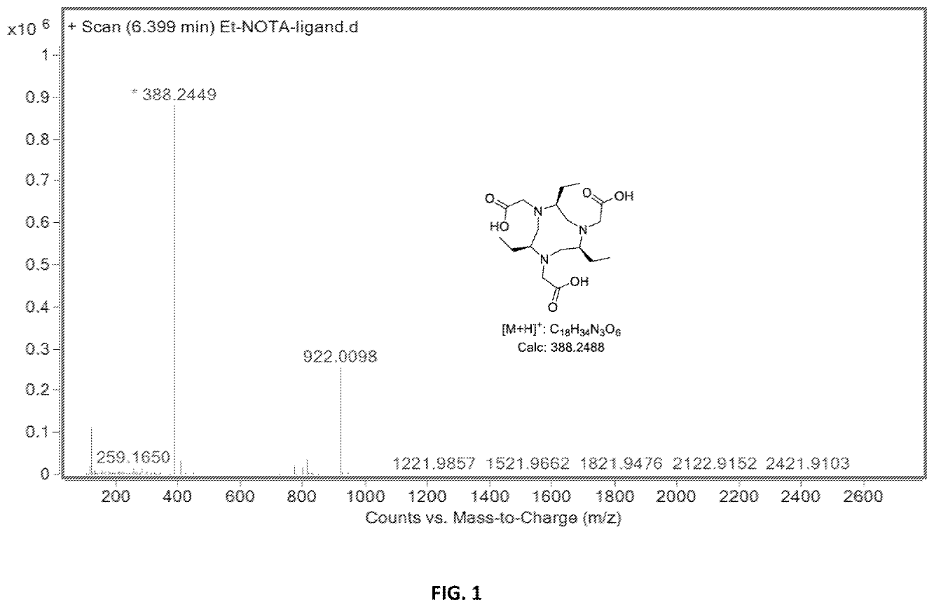

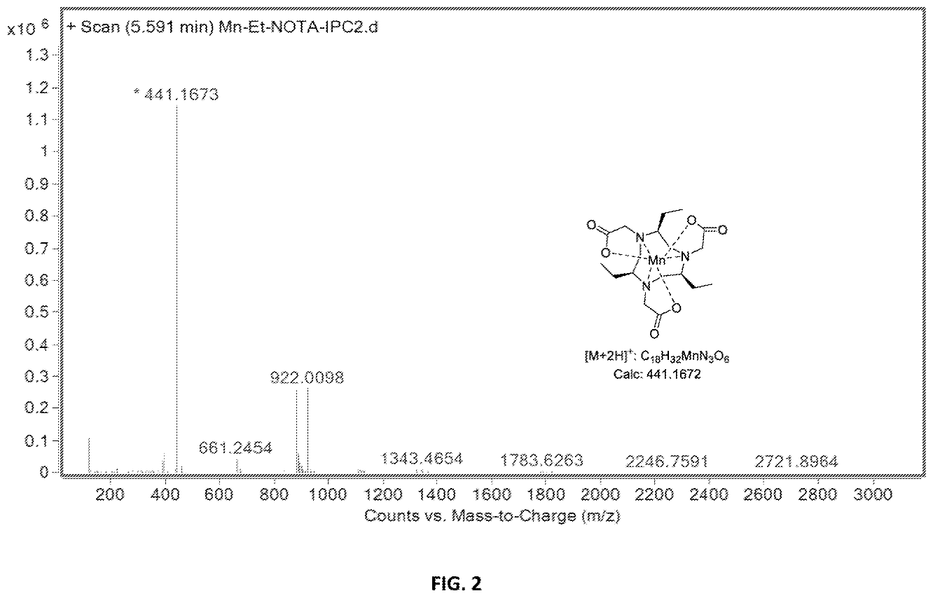

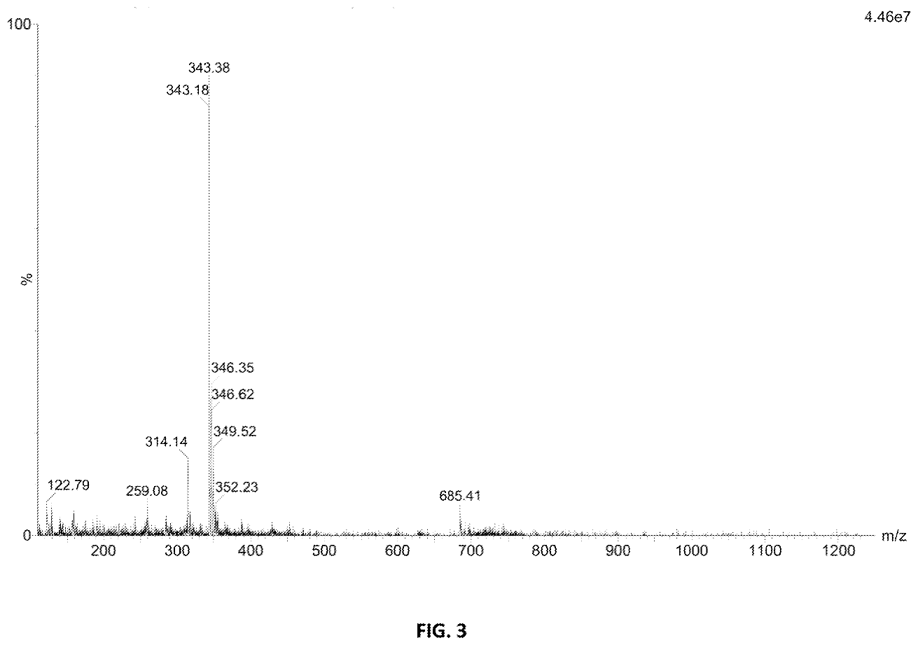

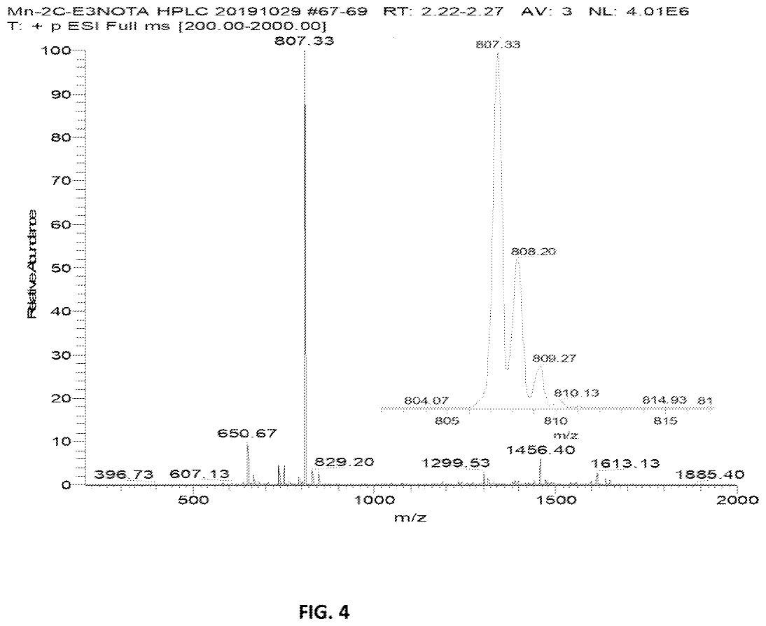

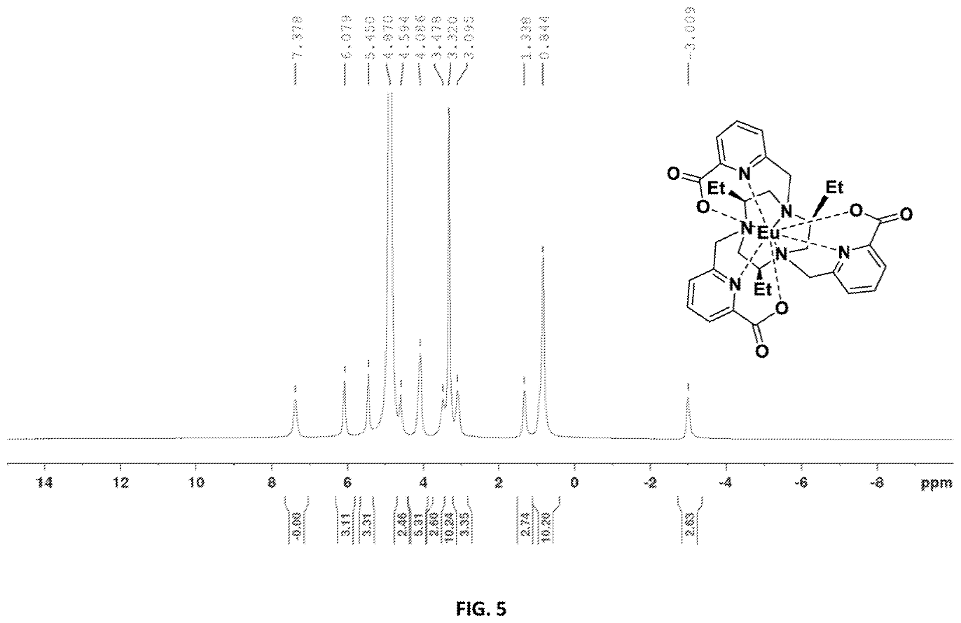

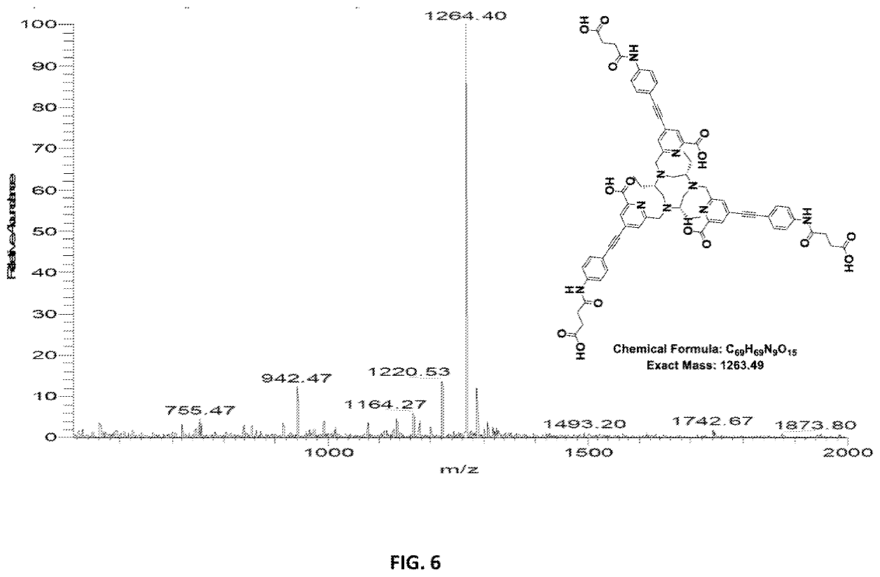

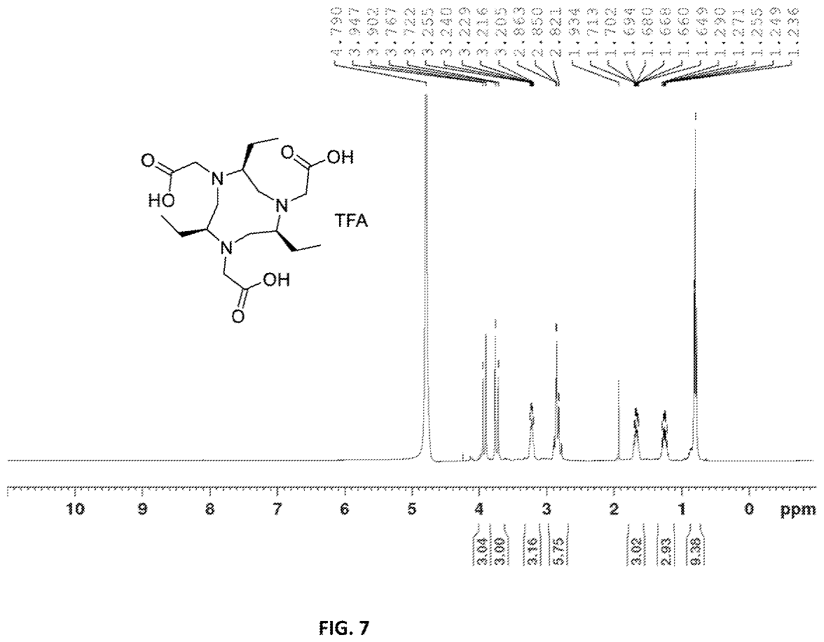

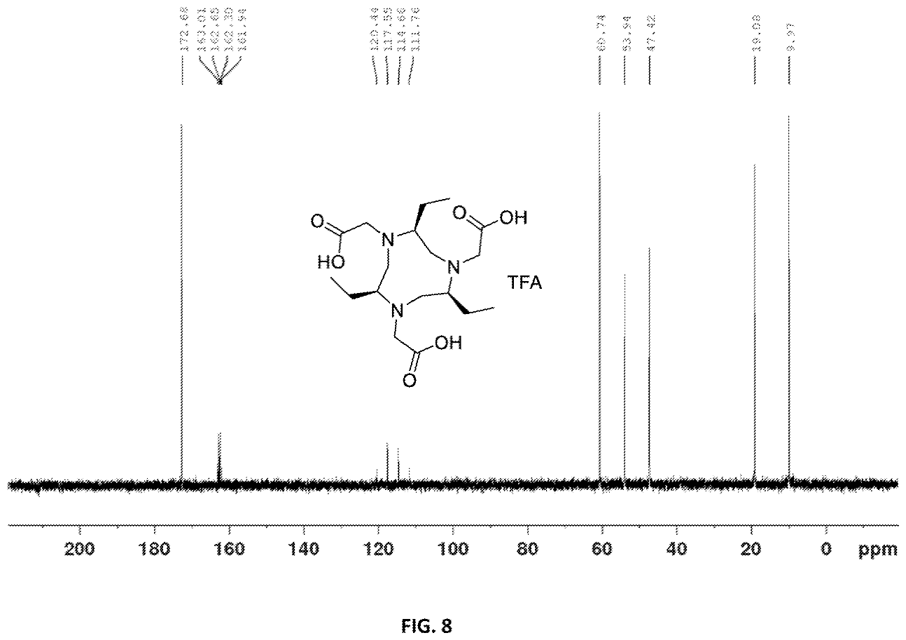

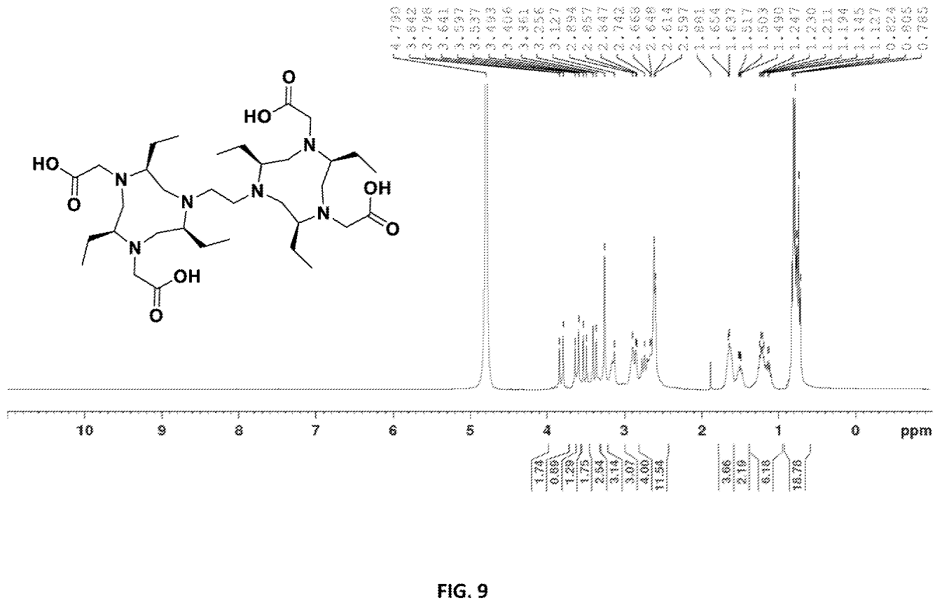

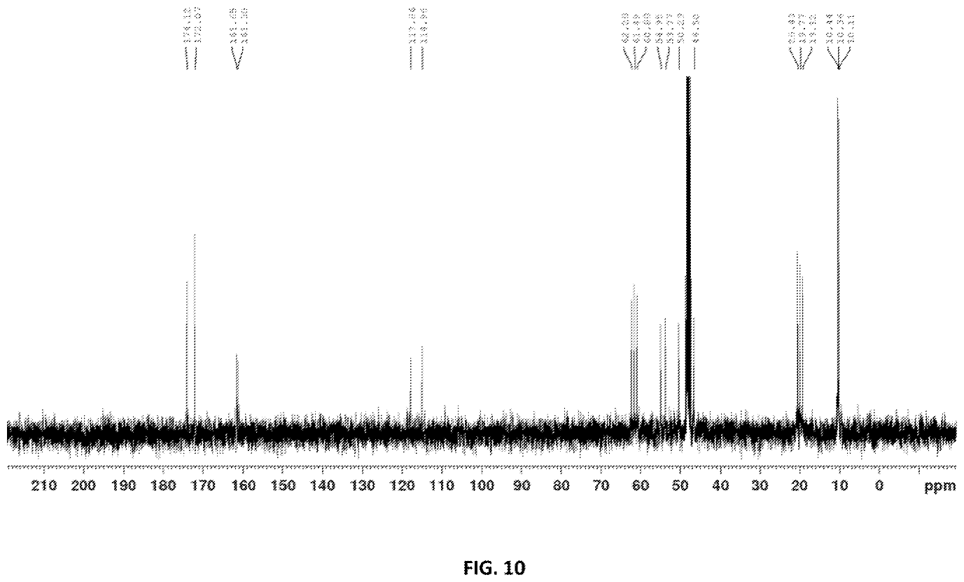

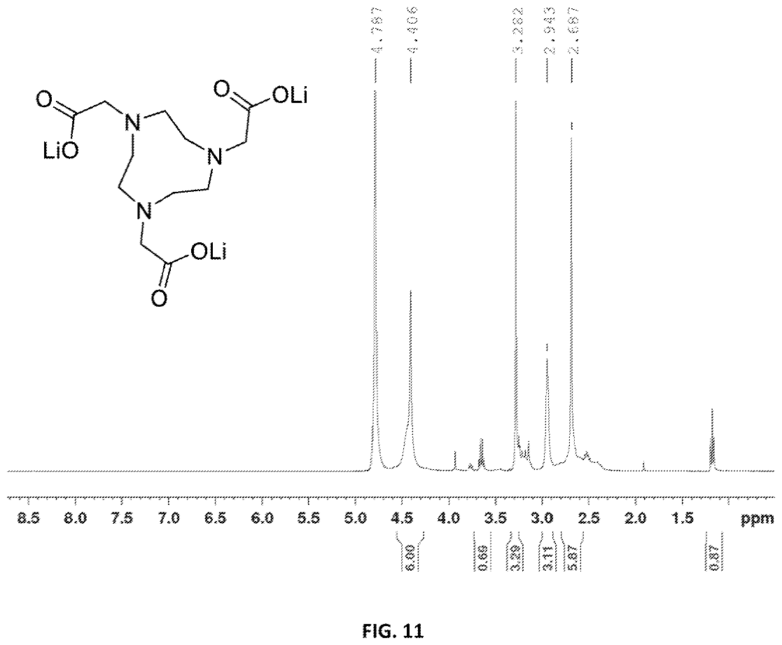

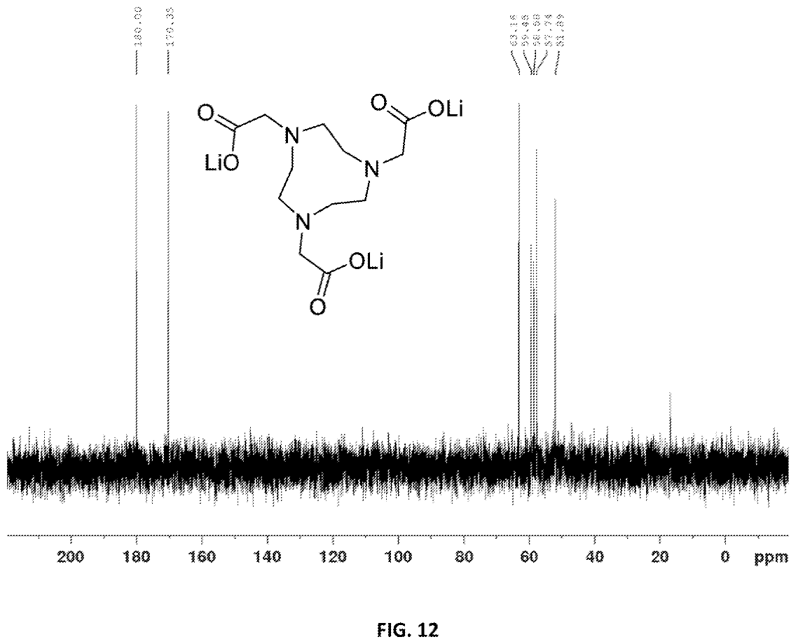

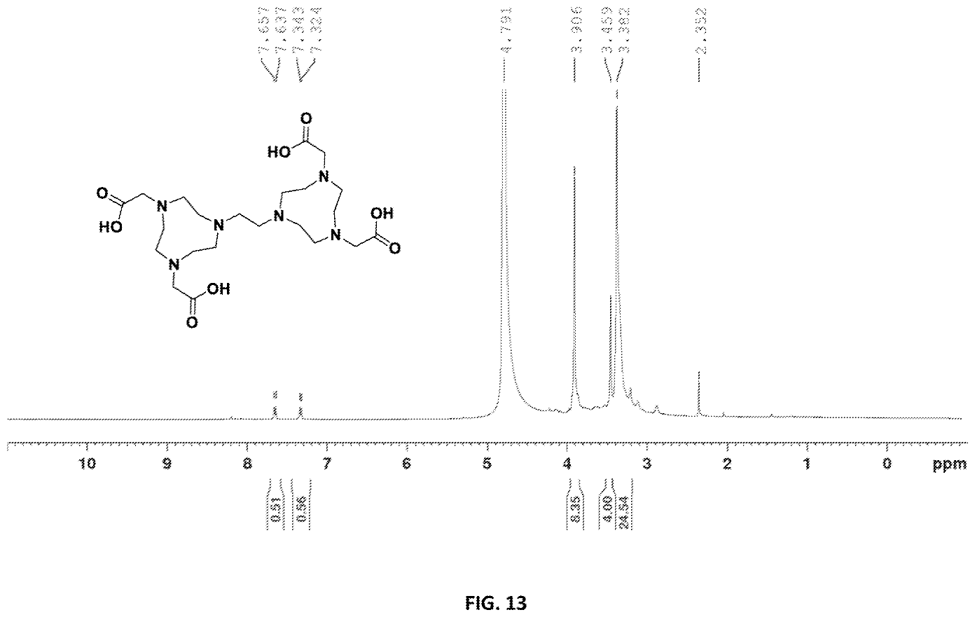

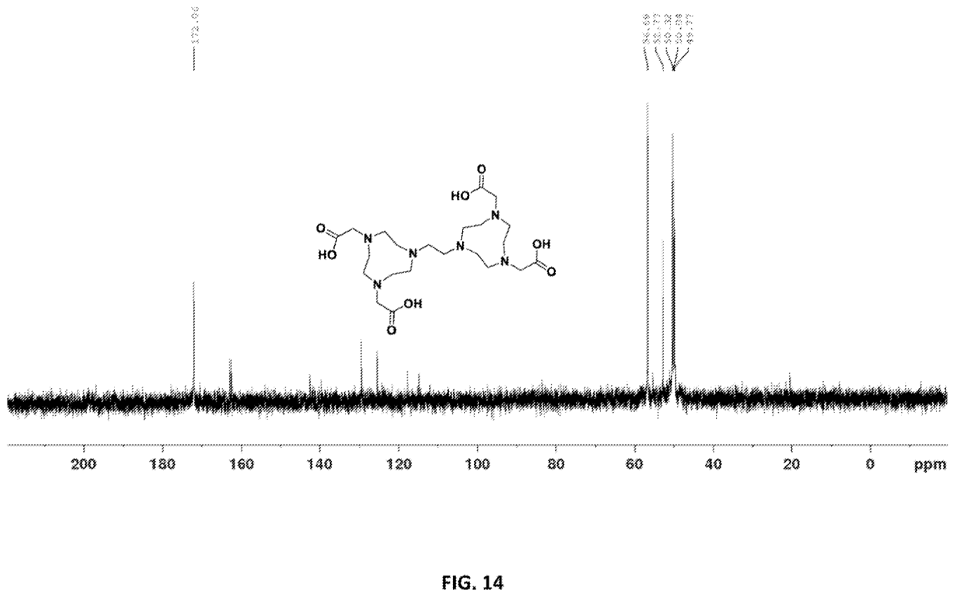

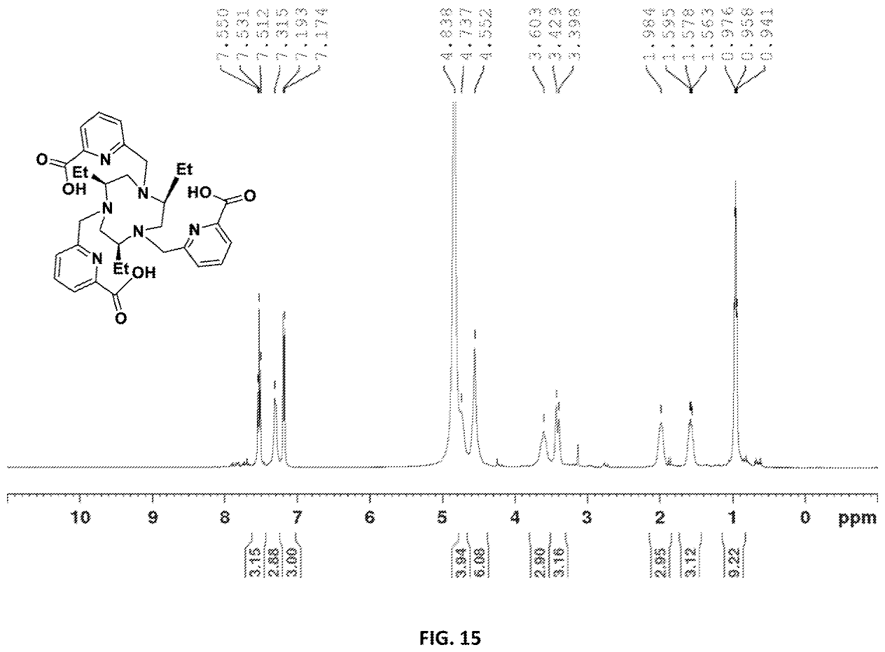

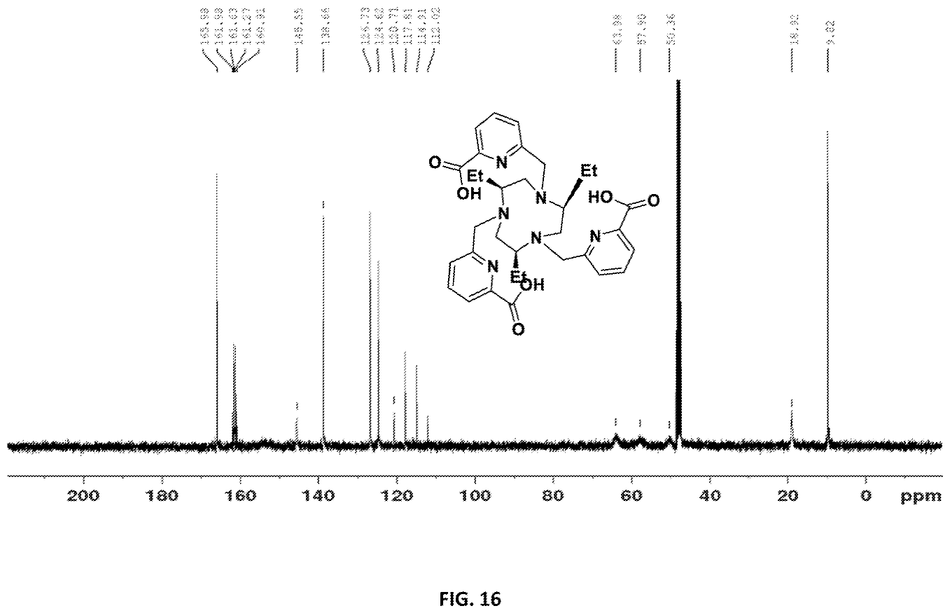

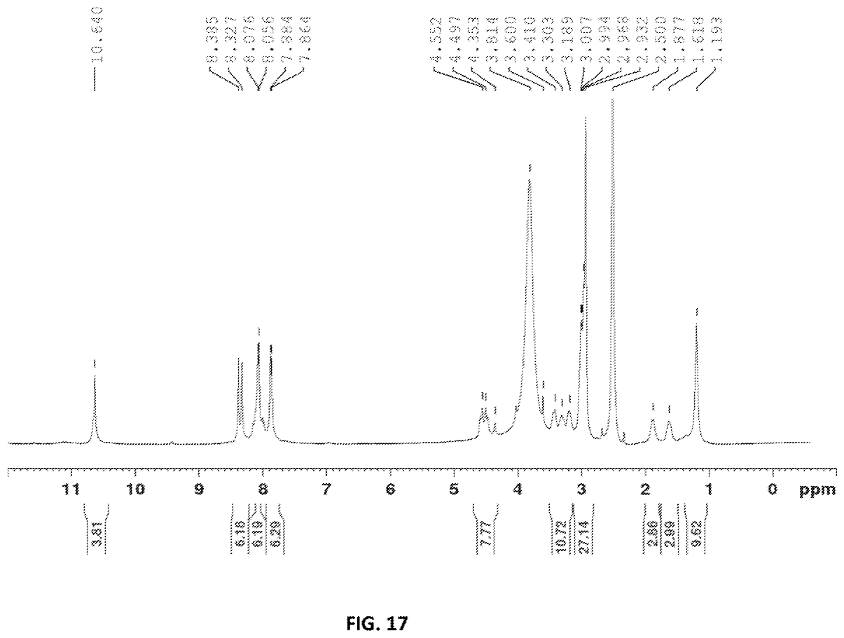

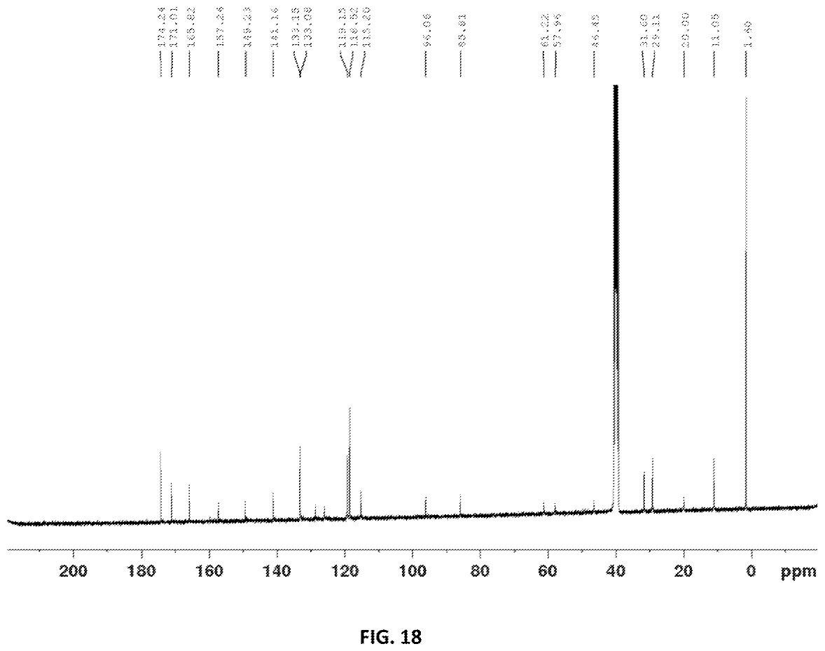

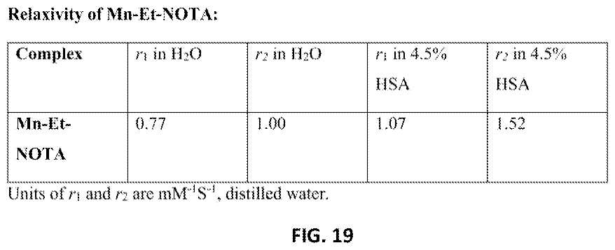

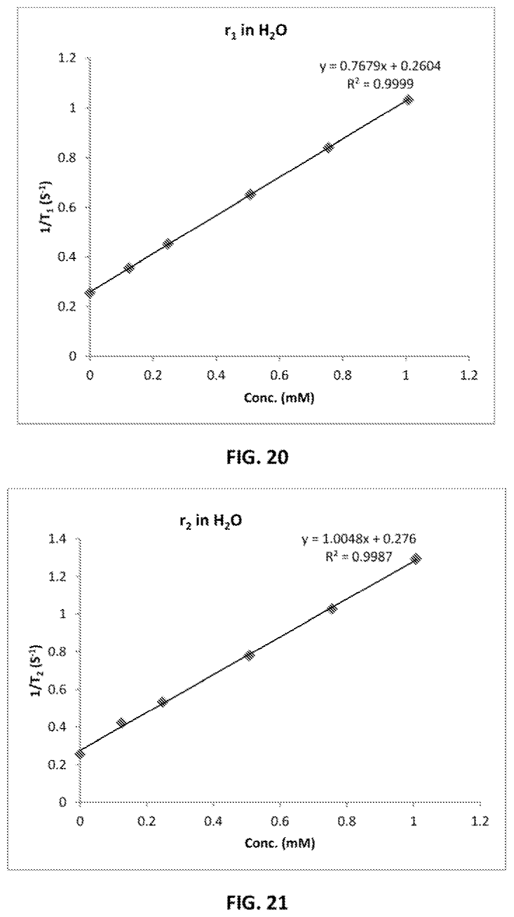

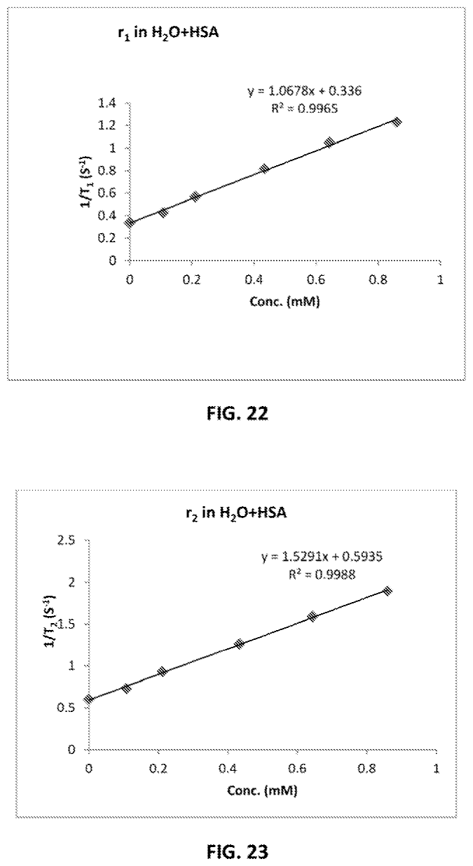

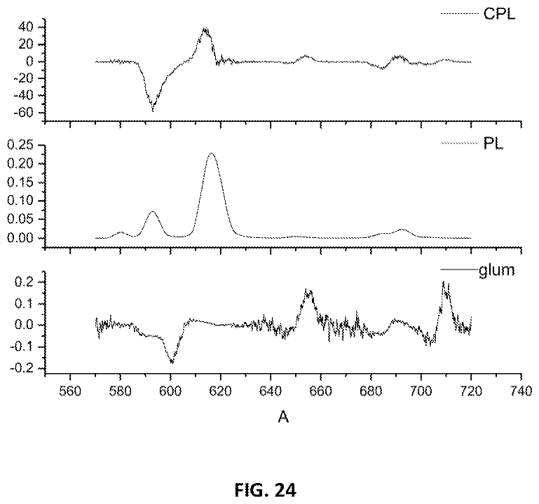

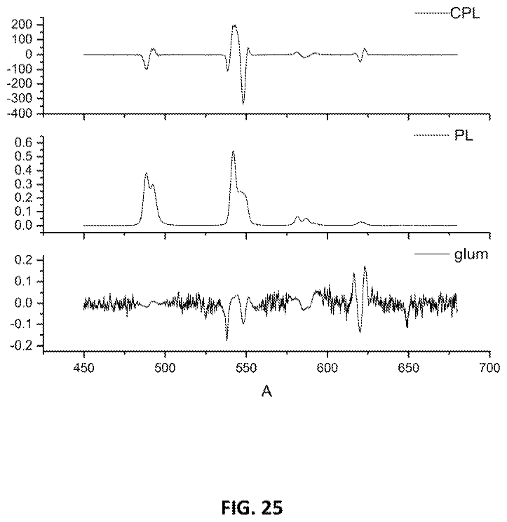

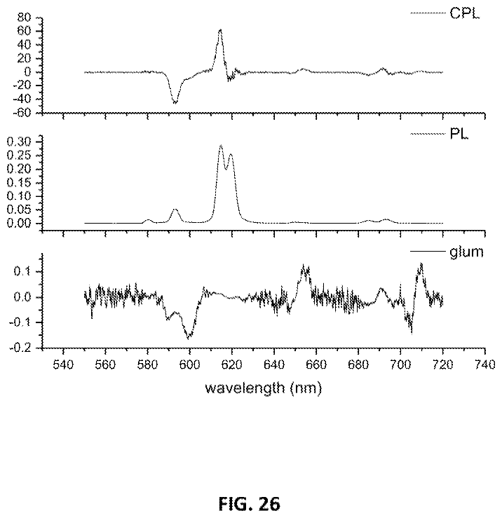

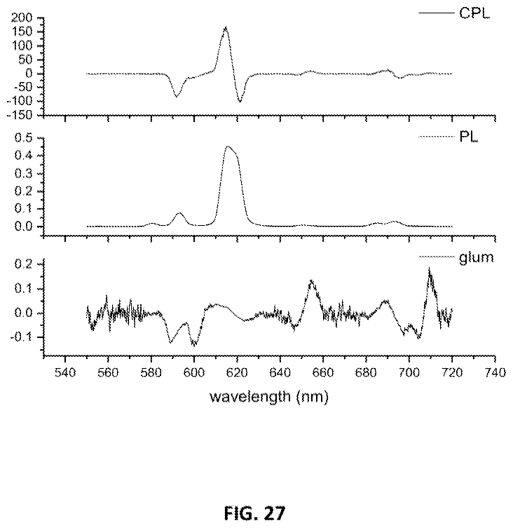

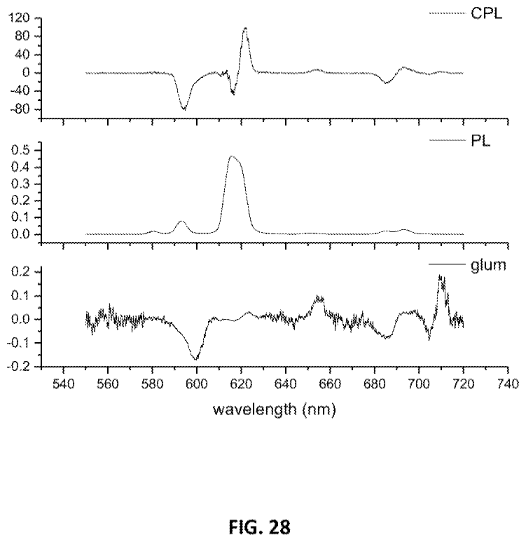

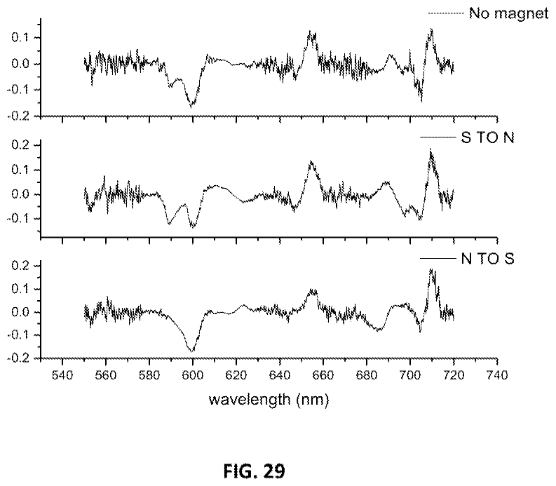

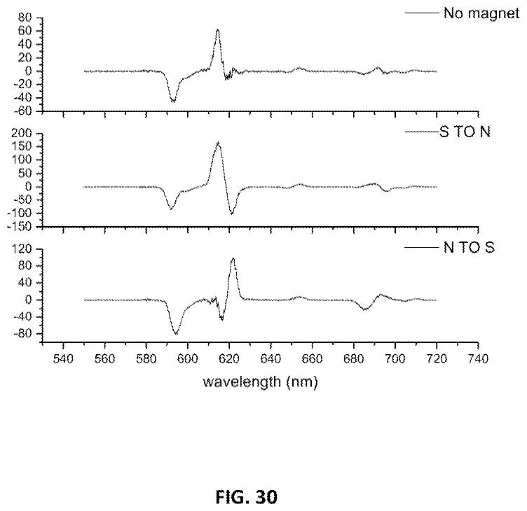

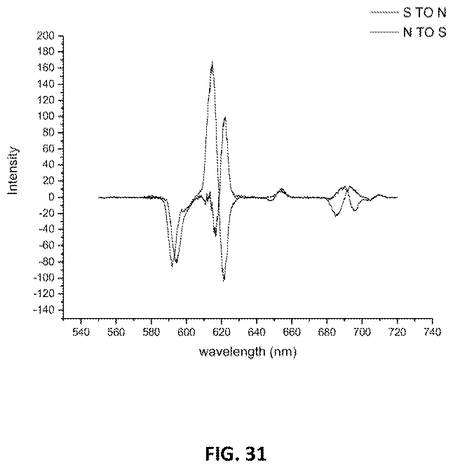

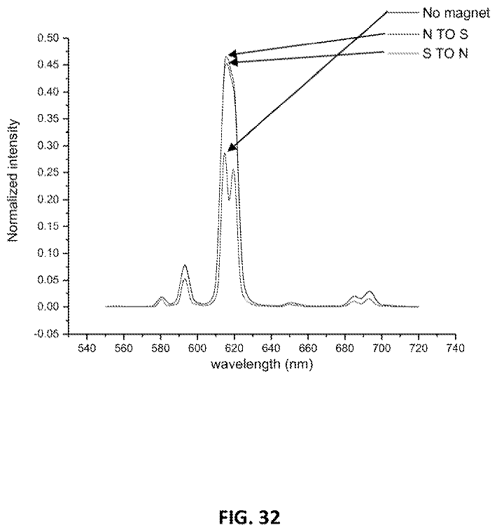

### DETAILED DESCRIPTION

Provided herein are chiral NOTA chelators and their metal complexes useful as MRI, PET contrast agents, and luminescent/CPL materials, their methods of use and preparation thereof.

As used herein, unless otherwise indicated, the term “halo” or “halide” includes fluoro, chloro, bromo or iodo. Preferred halo groups are fluoro, chloro and bromo.

As used herein, “alkyl” refers to a straight-chain or branched saturated hydrocarbon group. Examples of alkyl groups include methyl (Me), ethyl (Et), propyl (e.g., n-propyl and z′-propyl), butyl (e.g., n-butyl, z′-butyl, sec-butyl, tert-butyl), pentyl groups (e.g., n-pentyl, z′-pentyl, -pentyl), hexyl groups, and the like. In various embodiments, an alkyl group can have 1 to 40 carbon atoms (i.e., C1-40 alkyl group), for example, 1-30 carbon atoms (i.e., C1-30 alkyl group). In certain embodiments, an alkyl group can have 1 to 6 carbon atoms, and can be referred to as a “lower alkyl group.” Examples of lower alkyl groups include methyl, ethyl, propyl (e.g., n-propyl and z′-propyl), and butyl groups (e.g., n-butyl, z′-butyl, sec-butyl, tert-butyl). In certain embodiments, alkyl groups can be substituted as described herein. An alkyl group is generally not substituted with another alkyl group, an alkenyl group, or an alkynyl group.

As used herein, “alkenyl” refers to a straight-chain or branched alkyl group having one or more carbon-carbon double bonds. Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In various embodiments, an alkenyl group can have 2 to 40 carbon atoms (i.e., C2-40 alkenyl group), for example, 2 to 20 carbon atoms (i.e., C2-20 alkenyl group). In certain embodiments, alkenyl groups can be substituted as described herein. An alkenyl group is generally not substituted with another alkenyl group, an alkyl group, or an alkynyl group.

As used herein, “cycloalkyl” by itself or as part of another substituent means, unless otherwise stated, a monocyclic hydrocarbon having between 3-12 carbon atoms in the ring system and includes hydrogen, straight chain, branched chain, and/or cyclic substituents. Exemplary cycloalkyls include cyclopropyl, cyclobutyl, cyclopentyl, cyclohexyl, cycloheptyl, and the like.

As used herein, a “fused ring” or a “fused ring moiety” refers to a polycyclic ring system having at least two rings where at least one of the rings is aromatic and such aromatic ring (carbocyclic or heterocyclic) has a bond in common with at least one other ring that can be aromatic or non-aromatic, and carbocyclic or heterocyclic. These polycyclic ring systems can be highly p-conjugated and optionally substituted as described herein.

As used herein, “heteroatom” refers to an atom of any element other than carbon or hydrogen and includes, for example, nitrogen, oxygen, silicon, sulfur, phosphorus, and selenium.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or cycloheteroalkyl rings. An aryl group can have 6 to 24 carbon atoms in its ring system (e.g., C6-24 aryl group), which can include multiple fused rings. In certain embodiments, a polycyclic aryl group can have 8 to 24 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In certain embodiments, aryl groups can be optionally substituted. In certain embodiments, an aryl group can have one or more halogen substituents, and can be referred to as a “haloaryl” group. Perhaloaryl groups, i.e., aryl groups where all of the hydrogen atoms are replaced with halogen atoms (e.g., —C₆F₅), are included within the definition of “haloaryl.” In certain embodiments, an aryl group is substituted with another aryl group and can be referred to as a biaryl group. Each of the aryl groups in the biaryl group can be optionally substituted.

The term “aralkyl” refers to an alkyl group substituted with an aryl group.

As used herein, “heteroaryl” refers to an aromatic monocyclic ring system containing at least one ring heteroatom selected from oxygen (O), nitrogen (N), sulfur (S), silicon (Si), and selenium (Se) or a polycyclic ring system where at least one of the rings present in the ring system is aromatic and contains at least one ring heteroatom. Polycyclic heteroaryl groups include those having two or more heteroaryl rings fused together, as well as those having at least one monocyclic heteroaryl ring fused to one or more aromatic carbocyclic rings, non-aromatic carbocyclic rings, and/or non-aromatic cycloheteroalkyl rings. A heteroaryl group, as a whole, can have, for example, 5 to 24 ring atoms and contain 1-5 ring heteroatoms (i.e., 5-20 membered heteroaryl group). The heteroaryl group can be attached to the defined chemical structure at any heteroatom or carbon atom that results in a stable structure. Generally, heteroaryl rings do not contain O—O, S—S, or S-0 bonds. However, one or more N or S atoms in a heteroaryl group can be oxidized (e.g., pyridine Noxide thiophene S-oxide, thiophene S,S-dioxide). Examples of heteroaryl groups include, for example, the 5- or 6-membered monocyclic and 5-6 bicyclic ring systems shown below: where T is O, S, NH, N-alkyl, N-aryl, N-(arylalkyl) (e.g., N-benzyl), SiH₂, SiH(alkyl), Si(alkyl)₂, SiH(arylalkyl), Si(arylalkyl)₂, or Si(alkyl)(arylalkyl). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In certain embodiments, heteroaryl groups can be substituted as described herein. In certain embodiments, heteroaryl groups can be optionally substituted.

The term “optionally substituted” refers to a chemical group, such as alkyl, cycloalkyl aryl, and the like, wherein one or more hydrogen may be replaced with a with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” and “sulfone” is art-recognized and refers to —SO₂—. “Halide” designates the corresponding anion of the halogens.

The term “pharmaceutically acceptable carrier” refers to a medium that is used to prepare a desired dosage form of a compound. A pharmaceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecyl sulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counterions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

Two chiral ligands were designed and synthesized based on a chiral triazacyclononane (TACN) used as the chiral macrocyclic backbone, where the chiral group can be optionally substituted to give the chiral NOTA compounds. To be consistent, they are referred to as Et-NOTA and Et-ENOTA. From the table shown below, it is supposed that the manganese complexes of NOTA and Et-NOTA without water molecule coordinated on the first-sphere of the metal ions, while the complexes of ENOTA and Et-ENOTA complexes are dimeric complexes, each of the chelating group with one water molecule coordinated on the metal ions. Therefore the relaxivity of the former two complexes would be expected to be lower compared to the latter two complexes, but the comparison of achiral and chiral NOTA complexes is also crucial for the development of NOTA-based MRI contrast agents and for the other potential applications.

Except for the chiral NOTA (E3NOTA), the chiral TACN was used to synthesize chiral ligands of lanthanides. Three types of chromophores were designed around the chiral TACN cyclic backbone. This would just obtain one isomer after complexation, it was hypothesized that the chiral complexes with both higher stability and rigidity would make them promising complexes for CPL applications.

The chiral NOTA macrocyclic compounds and their derivatives with and without a linker, with a bioconjugatable handle that can be attached to, e.g., vectors, peptides, proteins, small molecule, nanoparticles, and antibodies.

The chiral NOTA macrocyclic compounds described herein can be used as chelators for use as PET, CT and MRI contrast agents. These ligands are suitable to form complexes with gallium — used in PET or with and without nanoparticles for CT and also provides an alternative to MRI contrast agents that use gadolinium.

These chelators are suitable ligands for manganese, which is a potential metal to replace gadolinium as an MM contrast agent. This is important as there are concerns regarding the safety of gadolinium based contrast agents, which has limited the number of available commercial contrast agents available to be used on the market.

The present disclosure provides a chiral NOTA chelator of Formula 1:

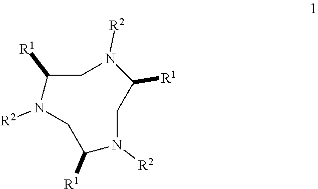

or a pharmaceutically acceptable salt or zwitterion thereof, wherein
R¹ is selected from the group consisting of hydrogen, alkyl, alkene, alkyne, cycloalkyl, heterocycloalkyl, aryl, heteroaryl, araalkyl, and —(CR₂)ₙY, wherein n is a whole number selected from 1-10; each R is independently hydrogen, alkyl, cycloalkyl, or aryl; or two R taken together with the carbon(s) to which they are attached form a 3-6 membered cycloalkyl; and Y is hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R¹ is a side chain of a naturally occurring amino acid or the side chain of a D-isomer of a naturally occurring amino acid; or R¹ is a moiety having the structure:

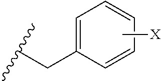

wherein X is azide, alkyne, halide, tosylate, mesylate, or hydroxyl; and
R² is —(C═O)OH, —(C═O)NHR⁵, or —(CH₂)ₘZ, wherein m is a whole number selected from 2-8; R⁵ is a targeting agent; and Z is moiety of Formula 2:

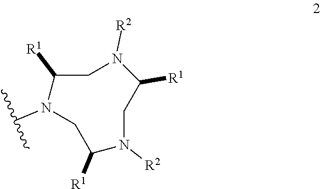

or a pharmaceutically acceptable salt or zwitterion thereof; or R² is a moiety of Formula 3:

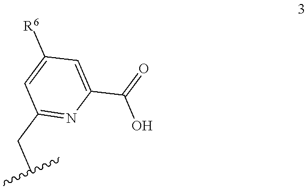

or a pharmaceutically acceptable salt or zwitterion thereof, wherein R⁶ for each occurrence is independently hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R⁶ is a moiety of Formula 4:

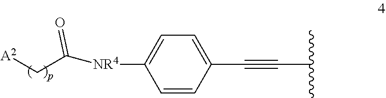

wherein p is a whole number selected from 1-6;
each A² is independently —CO₂R⁵, —NHR⁵, —OR⁵, N₃, or alkyne; and
R⁴ is hydrogen or alkyl, with the proviso that if one R² is —(CH₂)₂Z and four R² are each —(C═O)OH, then each R¹ cannot be hydrogen; and if three R² are each —(C═O)OH, then each R¹ cannot be hydrogen.

The proviso if one R² is —(CH₂)₂Z and four R² are each —(C═O)OH, then each R¹ cannot be hydrogen; and if three R² are each —(C═O)OH, then each R¹ cannot be hydrogen is intended to exclude the following NOTA complexes from the NOTA complex of Formula 1:

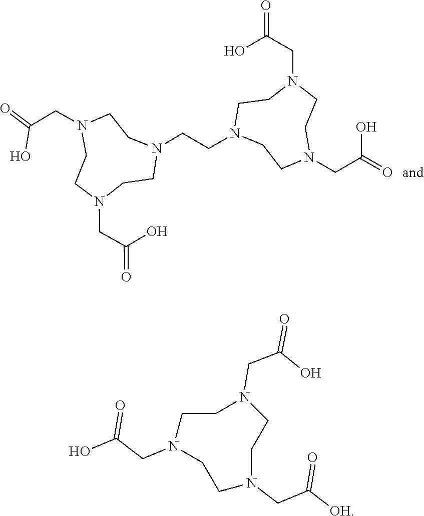 [SMILES: O=C(O)CN1CCN(CCN2CCN(CC(=O)O)CCN(CC(=O)O)CC2)CCN(CC(=O)O)CC1 ; O=C(O)CN1CCN(CC(=O)O)CCN(CC(=O)O)CC1]

The carbons covalently bonded to R¹ in the chiral NOTA chelators described herein are stereogenic centers and can thus exist as an S stereogenic center or as an R stereogenic center. For the purpose of clarity, only one relative stereogenic configuration of the chiral NOTA chelators is depicted. However, all relative and absolute stereogenic configurations of the chiral NOTA chelators described herein are contemplated by the present disclosure. In certain embodiments, the carbons covalently bonded to R¹ in the chiral NOTA chelator described herein are all S stereogenic centers or all R stereogenic centers.

In certain embodiments, the chiral NOTA chelator of Formula 1 has no more than one R⁵ group.

R¹ can be C₁-C₈ alkyl, C₁-C₇ alkyl, C₁-C₆ alkyl, C₁-C₅ alkyl, C₁-C₄ alkyl, C₁-C₃ alkyl, aryl, heteroaryl, or —(CR₂)ₙY, wherein n is a whole number selected from 1-8, 1-6, 1-4, 1-3, or 1-2, and Y is aryl or heteroaryl. In certain embodiments, Y is optionally substituted phenyl or optionally substituted indole, such as an optionally substituted 3-indole.

In certain embodiments, R¹ is methyl, ethyl, propyl, butyl, pentyl, or hexyl; or 3-(λ³-methyl)-1H-indole shown below:

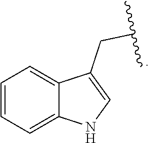 [SMILES: *Cc1c[nH]c2ccccc12]

In certain embodiments, R¹ is the side chain of a naturally occurring amino acid or the side chain of a D-isomer of a naturally occurring amino acid.

In certain embodiments, each R² is —(C═O)OH; or each R² is Formula 3.

In certain embodiments, the chiral NOTA chelator of Formula 1 has no more than one moiety of Formula 2.

In certain embodiments, R³ is hydrogen, alkyl, or aryl.

In certain embodiments, p is a whole number selected from 1-5, 1-4, 1-3, 1-2, 2-6, 2-5, 2-4, 2-4, or 2-3.

The targeting agent may selectively direct and bind the chiral NOTA chelator of Formula 1 and metal complexes comprising the same to a tissue type, a cell type, a cellular organelle, a binding partner, such as a cell surface receptor or a ligand, a nucleic acid sequence, or an infectious agent. The targeting agent can be a protein, glycoprotein, a glycolipid, a peptide, an antibody, an antibody fragment, an aptamer, or a small molecule. The chiral NOTA chelator of Formula 1 can be directly attached to the targeting agent or by a chemical linker.

In instances where the chiral NOTA chelator of Formula 1 is attached to the targeting agent via a linker, any linker in the art can be used to attach the chiral NOTA chelator of Formula 1 and the targeting agent. The selection of the linker is well within the skill of a person skilled in the art. Exemplary linkers include, but are not limited to polyethylene glycol linkers, alkyl amides, alkyl esters, alkyl sulfonamides, alkyl sulfones, alkanes, aryl amides, aryl esters, aryl sulfonamides, aryl sulfones, aryl, and combinations thereof. The linker can be covalently attached to the targeting agent by an amide bond, ester bond, sulfone bond, urea bond, ether bond or the like.

In certain embodiments, the chiral NOTA complex has the Formula 5 or Formula 6:

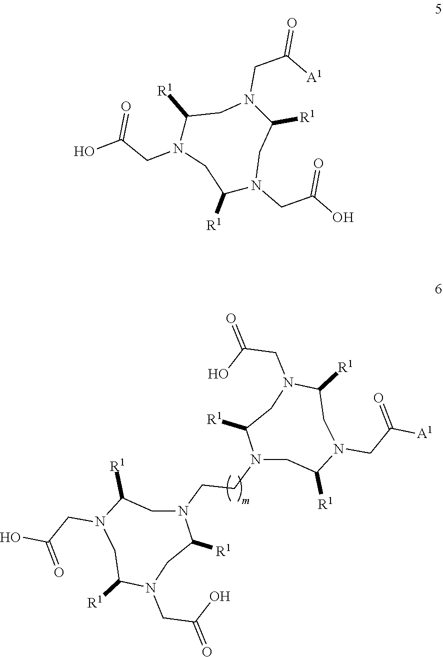

or a pharmaceutically acceptable salt or zwitterion thereof, wherein
m is a whole number selected from 2-8;
A¹ is OH or NHR⁵;
R¹ is selected from the group consisting of hydrogen, alkyl, alkene, alkyne, cycloalkyl, heterocycloalkyl, aryl, heteroaryl, araalkyl, and —(CR₂)ₙY, wherein n is a whole number selected from 1-10; each R is independently hydrogen, alkyl, cycloalkyl, or aryl; or two R² taken together with the carbon(s) to which they are attached form a 3-6 membered cycloalkyl; and Y is hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R¹ is a side chain of a naturally occurring amino acid or the side chain of a D-isomer of a naturally occurring amino acid; or R¹ is a moiety having the structure:

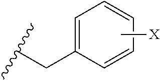

wherein X is azide, alkyne, halide, tosylate, mesylate, or hydroxyl; and
R⁵ is a targeting agent.

In certain embodiments, the chiral NOTA chelator has the Formula 7 or 8:

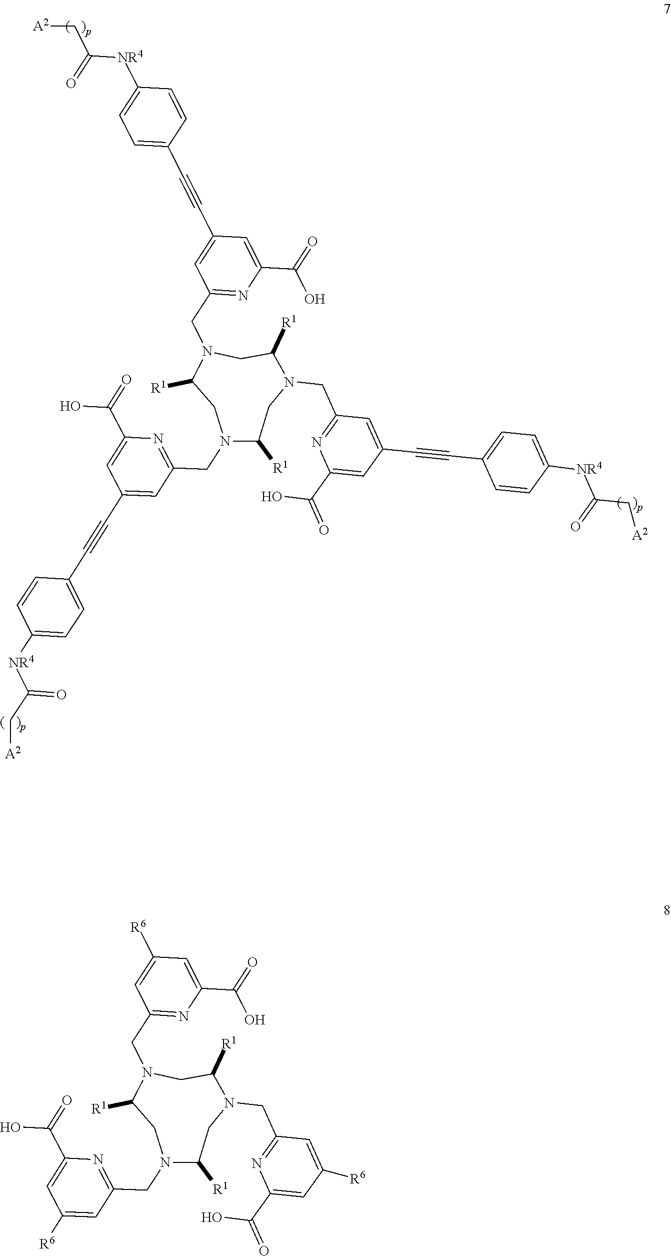

or a pharmaceutically acceptable salt or zwitterion thereof, wherein
p is a whole number selected from 1-6; and
each A² is independently —CO₂R⁵, —NHR⁵, —OR⁵, N₃, or alkyne;
R¹ is selected from the group consisting of hydrogen, alkyl, alkene, alkyne, cycloalkyl, heterocycloalkyl, aryl, heteroaryl, araalkyl, and —(CR₂)ₙY, wherein n is a whole number selected from 1-10; each R is independently hydrogen, alkyl, cycloalkyl, or aryl; or two R taken together with the carbon(s) to which they are attached form a 3-6 membered cycloalkyl; and Y is hydrogen, alkyl, alkene, alkyne, cycloalkyl, heterocycloalkyl, aryl, heteroaryl, cyano, halide, —N₃, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R¹ is a side chain of a naturally occurring amino acid or the side chain of a D-isomer of a naturally occurring amino acid; or R¹ is a moiety having the structure:

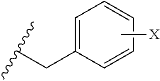

wherein X is azide, alkyne, halide, tosylate, mesylate, or hydroxyl;
R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl;
R⁴ is hydrogen or alkyl;
R⁵ is hydrogen or a targeting agent; and
R⁶ for each occurrence is independently hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl.

In certain embodiments, p is 2; R¹ is the side chain of a naturally occurring amino acid or the side chain of a D-isomer of a naturally occurring amino acid; and A² is —CO₂H.

In certain embodiments, the chiral NOTA chelator is selected from the group consisting of:

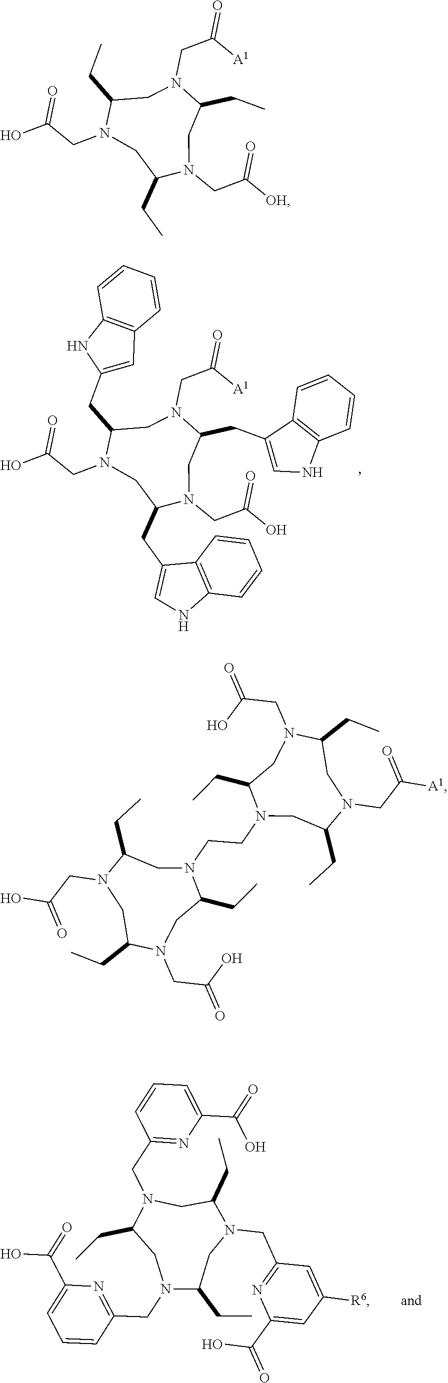

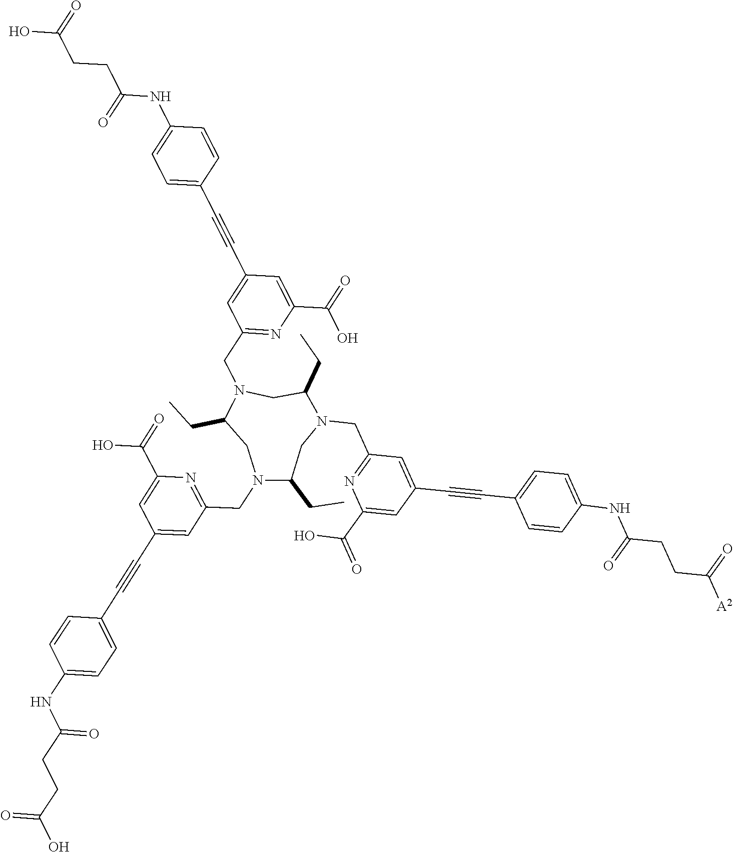

or a pharmaceutically acceptable salt or zwitterion thereof, wherein A¹ is OH or NHR⁵;
A² is OH or NHR⁵; and R⁶ is hydrogen or R⁵.

The present disclosure also provides a chiral NOTA complex comprising a chiral NOTA chelator described herein and at least one metal, wherein a metal complex is formed between the chiral NOTA complex and the at least one metal. The at least one metal can be selected from the group consisting of a paramagnetic metal and a positron emitting metal. In certain embodiments, the at least one metal is a Group 8-13 element of the periodic table, a lanthanide, or an actinide. In certain embodiments, the at least one metal is selected from the group consisting of aluminum, gallium, indium, iron, nickel, manganese, cobalt, chromium, yttrium, zirconium, zinc, copper, lanthanum, cerium, praseodymium, neodymium, promethium, samarium, europium, gadolinium, terbium, dysprosium, holmium, erbium, thulium ytterbium, lutetium, thorium, uranium, americium, curium, and berkelium. The chiral NOTA complex can comprise 1 or 2 metals. In certain embodiments, the chiral NOTA complex comprises 1 or 2 metals selected from the group consisting of gallium, copper, iron, manganese, and gadolinium. The at least one metal can exist in any oxidation state. In certain embodiments, the oxidation state of the at least one metal is 1⁺, 2⁺, 3⁺, 4⁺, 5⁺, or 6⁺. Exemplary metals, include but are not limited to, Eu³⁺, Gd³⁺, Tb³⁺, Dy³⁺, Ho³⁺, Nd³⁺, Sm³⁺, Cr³⁺, Fe³⁺, Co²⁺, Ni²⁺, Cu²⁺, Pr³⁺, Yb³⁺, Dy³⁺, La3+, Au³⁺, Pb²⁺, Bi³⁺, or Mn²⁺.

In certain embodiments, the at least one metal is a positron emitting metal, such as ⁴³K, ⁵²Fe, ⁵⁷Co, ⁶⁷Cu, ⁶⁷Ga, ⁶⁸Ga, ⁷⁷Br, ⁸¹Rb/^{81M}Kr, ^{87M}Sr, ^{99M}Tc, ¹¹¹In, ¹¹³In, ¹²⁷Cs, ¹²⁹Cs, ⁵²Mn, ¹⁹⁷Hg, ²⁰³Pb and ²⁰⁶Bi. In certain embodiments, the positron emitting metal is ⁵²Mn.

In certain embodiments, the chiral NOTA complex of Formula 9, 10, or 11:

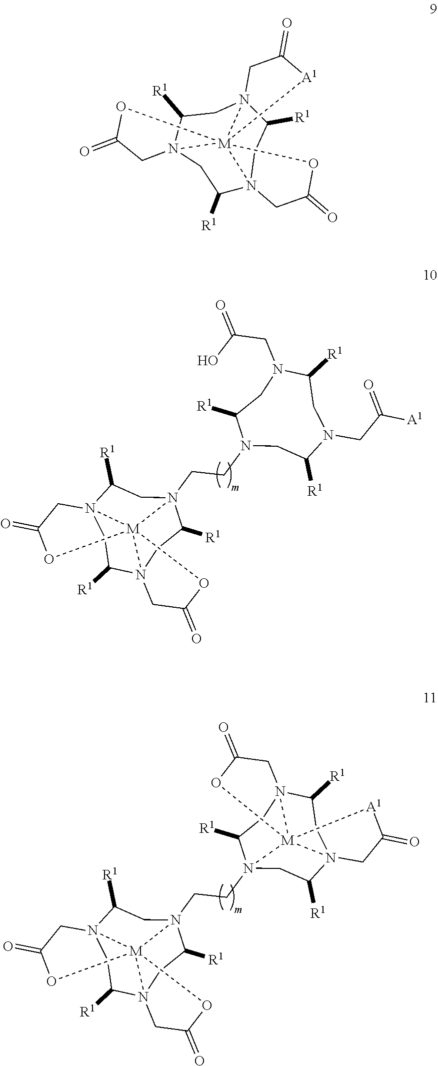

wherein M for each instance is independently a Group 8-13 element of the periodic table, a lanthanide, or an actinide; and m, R¹, and A¹ are independently as defined in any embodiment described herein. M can exist in any oxidations state, such as 1⁺, 2⁺, and 3⁺.

In certain embodiments, the chiral NOTA complex has the Formula 12 or 13:

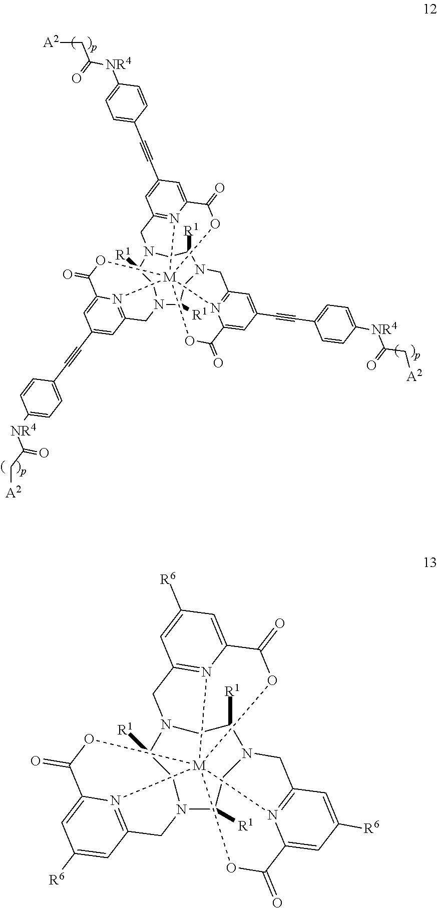

wherein M for each instance is independently a Group 8-13 element of the periodic table, a lanthanide, or an actinide; and m, R¹, R⁶, and A² are independently as defined in any embodiment described herein. In certain embodiments, M is Dy, Gd, Eu, Tm, Tb, Lu, Yb, Y, In, or Mn. M can be in any oxidation. Suitable oxidation states include, but are not limited to, 1⁺, 2⁺, and 3⁺.

The present disclosure also provides a pharmaceutical composition comprising any chiral NOTA complexes described herein and at least one pharmaceutically acceptable excipient.

The chiral NOTA complexes described herein and their pharmaceutically acceptable salts can be administered to a subject either alone or in combination with pharmaceutically acceptable carriers or diluents in a pharmaceutical composition according to standard pharmaceutical practice. The chiral NOTA complexes can be administered orally or parenterally, preferably parenterally. Parenteral administration includes intravenous, intramuscular, intraperitoneal, subcutaneous and topical, the preferred method being intravenous administration.

Accordingly, the present disclosure provides pharmaceutically acceptable compositions, which comprise a therapeutically-effective amount of one or more of the chiral NOTA complexes described herein, formulated together with one or more pharmaceutically acceptable carriers (additives) and/or diluents. The pharmaceutical compositions of the present disclosure may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; and (2) oral administration, for example, drenches (aqueous or non-aqueous solutions or suspensions), tablets, e.g., those targeted for buccal, sublingual, and systemic absorption, boluses, powders, granules, pastes for application to the tongue. The preferred method of administration of the chiral NOTA complexes of the present invention is parental administration (intravenous).

As set out herein, certain embodiments of the chiral NOTA complexes described herein may contain a basic functional group, such as amino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present disclosure. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the chiral NOTA complexes of the present disclosure include the conventional nontoxic salts or quaternary ammonium salts of the compounds, e.g., from nontoxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the chiral NOTA complexes described herein may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of the chiral NOTA complexes of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives, solubilizing agents, buffers and antioxidants can also be present in the compositions.

Methods of preparing these formulations of the chiral NOTA complexes include the step of bringing into association a chiral NOTA complex described herein with the carrier and, optionally, one or more accessory ingredients. In general, the formulations are prepared by uniformly and intimately bringing into association a chiral NOTA complex of the present invention with liquid carriers (liquid formulation), liquid carriers followed by lyophylization (powder formulation for reconstitution with sterile water or the like), or finely divided solid carriers, or both, and then, if necessary, shaping or packaging the product.

Pharmaceutical compositions of the present disclosure suitable for parenteral administration comprise one or more chiral NOTA complexes described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, chelating agents, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms upon the chiral NOTA complexes of the present disclosure may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The chiral NOTA complexes described herein can be used for MRI, PET, CT, and CPL imaging, and diagnostic methods, and analyte detection and quantitation and can be attached to any targeting agent, such as peptides, proteins, nanoparticles or the like.

The chiral NOTA complexes described herein are useful for in vitro and in vivo imaging. In certain embodiments, the imaging is optical imaging, magnetic resonance imaging, positron emission tomography, and single photon emission computed tomography.

Provided herein is a method of imaging a sample, the method comprising contacting a sample with a chiral NOTA complex described herein, irradiating the sample with radiation, and detecting radiation emitted by the chiral NOTA complex.

The sample can be derived from or a biological sample obtained from a subject, wherein the biological sample is a stool, urine, saliva, cerebrospinal fluid, blood, serum, plasma, tissue, or lacrimal fluid. The sample may comprise a cell or tissue. The subject can be any animal including, but not limited to, humans, non-human primates, domesticated animals (e.g., cats, dogs, etc.), livestock (e.g., cattle, horses, pigs, sheep, goats, etc.), laboratory animals (e.g., mouse, rabbit, rat, guinea pig, etc.), and birds.

The step of irradiating the sample can comprise irradiating with radiation selected from the group consisting of the radiation is visible to near infrared, radiowaves, high energy γ rays, lower energy γ rays, alpha particles, beta minus (electron emission), beta plus (positron emission) and gamma emitting radioisotopes, magnetic resonance and fluorescence.

The present disclosure also provides a method of imaging a subject, the method comprising administering a chiral NOTA complex described herein to the subject, irradiating the subject with radiation, and detecting radiation emitted by the chiral NOTA complex.

The subject can be any animal including, but not limited to, humans, non-human primates, domesticated animals (e.g., cats, dogs, etc.), livestock (e.g., cattle, horses, pigs, sheep, goats, etc.), laboratory animals (e.g., mouse, rabbit, rat, guinea pig, etc.), and birds.

The step of irradiating the subject can comprise irradiating with radiation selected from the group consisting of the radiation is visible to near infrared, radiowaves, high energy γ rays, lower energy γ rays, alpha particles, beta minus (electron emission), beta plus (positron emission) and gamma emitting radioisotopes, magnetic resonance and fluorescence.

The method of imaging a subject can comprise irradiating a target organ and detecting radiation emitted by the chiral NOTA complex. The target organ can be any organ in the subject including, but not limited to, a brain, a heart, a kidney, a liver, a lung, a plasma, or a spleen.

In certain embodiments, the chiral NOTA complex is administered orally, nasally, aurally, ocularly, sublingually, buccally, systemically, transdermally, mucosally, via cerebral spinal fluid injection, vein injection, muscle injection, peritoneal injection, or subcutaneous injection.

Also provided herein is the chiral NOTA complex described herein for use in imaging a sample. The present disclosure also provides the chiral NOTA complex described herein for use in imaging a subject. The present disclosure also provides the use of the chiral NOTA complex described herein in the manufacture of an imaging agent for imaging a subject.

In certain embodiment, provided herein is a method of using the chiral NOTA complex described herein for imaging, comprising the steps of (a) administering the chiral NOTA complex to a subject in need thereof; (b) detecting radiation emitted by the chiral NOTA complex; and (c) forming an image therefrom. In certain embodiments, the present disclosure provides a method of using the chiral NOTA complex described herein for imaging, comprising the steps of (a) administering the chiral NOTA complex to a subject in need thereof; (b) allowing sufficient time to permit the chiral NOTA complex to distribute within the subject; (c) exposing the subject to electromagnetic radiation absorbable by the chiral NOTA complex; (d) detecting radiation emitted by the metal complex; and (e) forming an image therefrom. In certain embodiments, the radiation is visible to near infrared, radiowaves, high energy γ rays, lower energy γ rays, alpha particles, beta minus (electron emission), beta plus (positron emission) and gamma emitting radioisotopes, magnetic resonance and fluorescence.

In certain embodiments, the present disclosure provides a method of using the chiral NOTA complex comprising the steps of (a) contacting a target with the chiral NOTA complex; (b) detecting radiation emitted by the chiral NOTA complex, and (c) measuring the amount and/or concentration of the chiral NOTA complex in the target. In certain embodiments, the imaging is fluorescent microscopy, flow cytometry, immunohistochemistry, immunoprecipitation, in situ hybridization and Forster resonance energy transfer. In certain embodiments, the target is blood or blood serum, bodily fluids, urine, feces, sputum, saliva amniotic fluid, duodenal fluid, cerebrospinal fluid, tissue biopsy, cell, cell extract, organ and tissue. In certain embodiments, the method is used in in vitro imaging. In certain embodiments, the radiation is visible to near infrared, radiowaves, high energy γ rays, lower energy γ rays, alpha particles, beta minus (electron emission), beta plus (positron emission) and gamma emitting radioisotopes, magnetic resonance and fluorescence.

Chiral NOTA complexes described herein can exhibit a number of improved properties as compared with conventional imaging agents, such as extended half-life, improved biodistribution and localization to specific organs, such as the brain, heart, kidney, liver, lung, plasma, or spleen, and enhanced relaxivities. By modifying chiral chiral NOTA complexes, a trend in the CPL properties can be observed, which indicates the chiral NOTA complexes potential for recognition of chiral biomolecules.

Chiral NOTA complexes of Formula 11 are capable of binding two metals and thus can used for dual imaging modalities to be used, such as PET/MRI. In such embodiments, the chiral NOTA complexes of Formula 11 can comprise a positron emitter, such as ⁴³K, ⁵²Fe, ⁵⁷Co, ⁶⁷Cu, ⁶⁷Ga, ⁶⁸Ga, ⁷⁷Br, ⁸¹Rb/^{81M}Kr, ^{87M}Sr, ^{99M}Tc, ¹¹¹In, ¹²³I, ¹²⁵I, ¹²⁷Cs, ¹²⁹Cs, ¹³¹I, ¹³²I, ⁵²Mn, ¹⁹⁷Hg, ²⁰³Pb and ²⁰⁶Bi; and a paramagnetic metal, such as Eu³⁺, Gd³⁺, Tb³⁺, Dy³⁺, Ho³⁺, Nd³⁺, Sm³⁺, Cr³⁺, Fe³⁺, Co²⁺, Ni²⁺, Cu²⁺, Pr³⁺, Yb³⁺, Dy³⁺, La3+, Au³⁺, Pb²⁺, Bi³⁺, or Mn²⁺.

### EXAMPLES

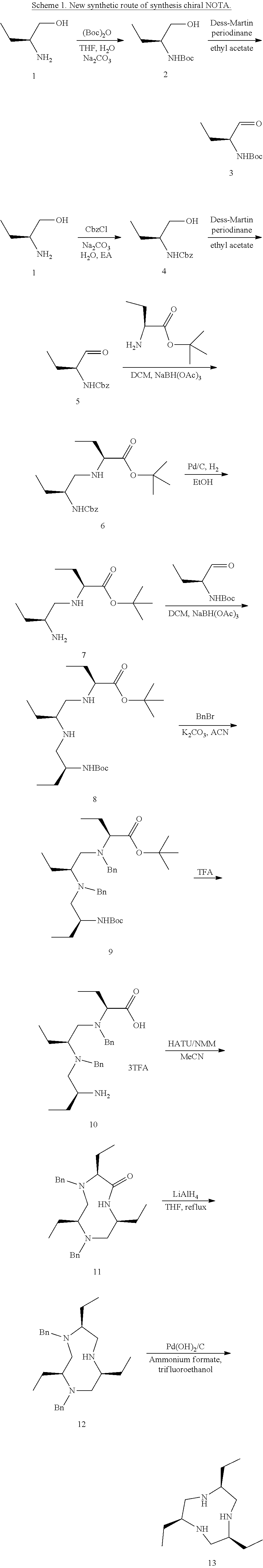

### Synthesis of Tert-butyl (S)-(1-hydroxybutan-2-yl)carbamate, 2

Compound (S)-2-aminobutan-1-ol (50 g, 0.56 mol) was dissolved in THF (300 mL) and water (300 mL), then Na₂CO₃ (119 g, 1.12 mol) was added, then into the reaction was added the solution of (Boc)₂O (135 g, 0.62 mol) in THF (200 mL) dropwise within 30 mins at room temperature, after stirring for 12 hours at room temperature, the mixture was extracted with ethyl acetate (400 mL×3), combined the organic layers and washed with brine (200 mL×1), dried over Na₂SO₄, filtered and the filtration was concentrated in vacuum, the obtained light yellow oil (108 g) was used to the next step reaction directly without further purification. ¹H NMR (400 MHz, Chloroform-d) δ 4.825 (s, 1H), 4.09 (d, J=7.2 Hz, 1H), 3.51-3.49 (m, 2H), 1.51-1.48 (m, 2H), 1.40 (s, 9H), 0.904 (t, J=7.6 Hz, 3H). ¹³C NMR (100 MHz, CDCl₃) δ 156.60, 79.45, 65.17, 54.16, 28.36, 24,46, 10.48.

### Synthesis of Tert-butyl (S)-(1-oxobutan-2-yl)carbamate, 3

Compound 2 (108 g, 0.56 mol) was dissolved in ethyl acetate (900 mL), then Dess-Martin periodinane (363 g, 0.84 mol) was added slowly at 0-20° C. with ice/water bath. The resulting mixture was stirred at this temperature for 3 hours, then filtered and the filter cake was washed with ethyl acetate (500 mL), the filtrate was concentrated in vacuum, the obtained light yellow oil (100 g, yield 95% for two steps) was used to the next step reaction directly without further purification.

¹H NMR (400 MHz, Chloroform-d) δ 9.54 (s, 1H), 5.16 (s, 1H), 4.14 (d, J=6.0 Hz, 1H), 1.93-1.86 (m, 1H), 1.65-1.56 (m, 1H), 1.40 (s, 9H), 0.92 (t, J=7.5 Hz, 3H). ¹³C NMR (100 MHz, CDCl₃) δ 200.00, 155.57, 79.94, 60.87, 28.25, 22.32, 9.42.

### Synthesis of Benzyl (S)-(1-hydroxybutan-2-yl)carbamate, 4

Compound (S)-2-aminobutan-1-ol (50 g, 0.56 mol) was dissolved in H₂O (400 mL), then Na₂CO₃ (119 g, 1.12 mol) was added, then into the reaction was added the solution of CbzCl (105 g, 0.62 mol) in ethyl acetate (100 mL) dropwise within 30 mins at 0-10° C., after stirring for 12 hours at room temperature, the mixture was extracted with ethyl acetate (400 mL×3), combined the organic layers and washed with brine (200 mL×1), dried over Na₂SO₄, filtered and the filtration was concentrated in vacuum, the obtained light yellow oil (120 g) was used to the next step reaction directly without further purification.

### Synthesis of Benzyl (S)-(1-oxobutan-2-yl)carbamate, 5

Compound 4 (120 g, 0.54 mol) was dissolved in ethyl acetate (1 L), then Dess-Martin periodinane (363 g, 0.84 mol) was added slowly at 0-20° C. with ice/water bath. The resulting mixture was stirred at this temperature for 3 hours, then filtered and the filter cake was washed with ethyl acetate (500 mL), the filtrate was concentrated in vacuum, the obtained light yellow oil (120 g, yield 96% for two steps) was used to the next step reaction directly without further purification. ¹H NMR (400 MHz, Chloroform-d) δ 9.57 (s, 1H), 7.38-7.26 (m, 5H), 5.42 (s, 1H), 5.12 (s, 2H), 4.29-4.12 (m, 1H), 2.01-1.94 (m, 1H), 1.73-1.66 (m, 1H), 0.95 (t, J=7.5 Hz, 3H). ¹³C NMR (100 MHz, CDCl₃) δ 199.30, 156.09, 136.17, 128.58, 128.27, 128.14, 67.10, 61.25, 22.31, 9.31.

### Synthesis of Tert-butyl (S)-2-(((S)-2-(((benzyloxy)carbonyl)amino) butyl)amino) butanoate, 6

Compound 5 (30 g, 0.14 mol) was dissolved in dichloromethane (300 mL), then added tert-butyl (S)-2-aminobutanoate (22 g, 0.14 mol), the reaction mixture was stirred at room temperature for 1 hour, then added sodium triacetoxyborohyride (59 g, 0.28 mol) slowly, the resulting mixture was stirred at room temperature for overnight (16 hours), added water (500 mL) and extracted with dichloromethane (400 mL×3), combined the organic phases and washed with brine (200 mL×1), dried over Na₂SO₄, filtered and the filtration was concentrated in vacuum, the obtained oil was purified by silica gel column chromatography, eluted with petroleum ether/ethyl acetate (5:1-1:2), this resulted in the product as a colorless oil (39 g, 80%). ¹H NMR (400 MHz, Chloroform-d) δ 7.44-7.28 (m, 5H), 5.09 (s, 2H), 5.02-4.95 (m, 1H), 3.57 (q, J=7.5, 7.1 Hz, 1H), 3.00 (t, J=6.6 Hz, 1H), 2.73-2.68 (m, 1H), 2.51-2.46 (m, 1H), 1.74-1.50 (m, 2H), 1.45 (s, 9H), 0.93-0.88 (m, 6H). ¹³C NMR (100 MHz, CDCl₃) δ 174.70, 174.52, 156.35, 136.74, 128.48, 128.03, 127.99, 81.00, 66.50, 63.74, 52.91, 50.58, 28.12, 26.33, 25.76, 10.20, 10.11.

### Synthesis of Tert-butyl (S)-2-(((S)-2-aminobutyl)amino)butanoate, 7

Compound 6 (30 g, 82 mmol) was dissolved in ethanol (400 mL), then added Pd/C (loading 10 wt %, wet with 50% water) (3 g), then hydrogenation with a hydrogen balloon for 8 hours, filtered and the filtrate was concentrated in vacuum, this resulted in the product as a colourless oil (18.5 g, 98%). ¹H NMR (400 MHz, Chloroform-d) δ 2.95-2.91 (m, 1H), 2.73-2.54 (m, 2H), 2.14-2.09 (m, 1H), 1.64-1.47 (m, 4H), 1.41 (s, 9H), 0.90-0.85 (m, 6H). ¹³C NMR (100 MHz, CDCl₃) δ 175.00, 80.71, 63.88, 54.63, 53.06, 28.36, 28.09, 26.68, 10.41, 10.14.

### Synthesis of Tert-butyl (6S,9S,12S)-8,11-dibenzyl-6,9,12-triethyl-2,2-dimethyl-4-oxo-3-oxa-5,8,11-triazatridecan-13-oate, 9

Compound 7 (15.0 g, 65 mmol) was dissolved in dichloromethane (150 mL), then added compound 3 (13.4 g, 72 mmol), the reaction mixture was stirred at room temperature for 1 hour, then added sodium triacetoxyborohyride (27.0 g, 0.13 mol) slowly, the resulting mixture was stirred at room temperature for 5 hours, added water (400 mL) and extracted with dichloromethane (300 mL×3), combined the organic phases and washed with brine (150 mL×1), dried over Na₂SO₄, filtered and the filtration was concentrated in vacuum, the obtained oil was dissolved in acetonitrile (200 mL), added K2CO3 (27.0 g, 0.20 mol) and benzyl bromide (26.7 g, 0.16 mol), after stirring at 50° C. for 16 hours, added water (500 mL) and extracted with ethyl acetate (300 mL×3), combined the organic phases and washed with brine (200 mL×1), dried over Na₂SO₄, filtered and the filtration was concentrated in vacuum, the obtained oil was purified by silica gel column chromatography, eluted with petroleum ether/ethyl acetate (10:1-3:1), this resulted in the product as a colorless oil (29.6 g, two steps yield 78%). ¹H NMR (400 MHz, Chloroform-d) δ 7.59-6.92 (m, 10H), 3.89-3.86 (m, 1H), 3.72-3.69 (m, 1H), 3.56-3.42 (m, 3H), 3.17-3.06 (m, 1H), 2.84-2.75 (m, 1H), 2.52-2.31 (m, 4H), 1.68-1.66 (m, 2H), 1.55 -1.41 (m, 20H), 1.30-1.13 (m, 2H), 0.89-0.73 (m, 9H). ¹³C NMR (100 MHz, CDCl₃) δ 172.51, 155.80, 140.55, 140.10, 129.07, 129.04, 128.08, 128.01, 126.78, 126.74, 80.79, 77.25, 64.62, 60.08, 56.15, 55.82, 54.85, 50.01, 28.53, 28.44, 26.03, 23.23, 22.67, 12.03, 10.92, 10.06.

### Synthesis of (S)-2-(((S)-2-(((S)-2-aminobutyl)(benzyl)amino)butyl) (benzyl) amino)butanoic acid, 10

The mixture of compound 9 (25 g, 43 mmol) in trifluoroacetic acid (120 mL) was stirred at room temperature for 16 hours, concentrated and then added diethyl ether (200 mL), after stirring for 5 hour, filtered and filter cake was dried in vacuum, this resulted in the product as TFA salt (29 g, 94%). ¹H NMR (400 MHz, Methanol-d₄) δ 7.30-6.45 (m, 10H), 3.65-3.62 (m, 1H), 3.50-3.47 (m, 1H), 3.36-3.14 (m, 2H), 3.14-2.82 (m, 3H), 2.73-2.67 (m, 2H), 2.54-2.11 (m, 2H), 1.61-0.79 (m, 6H), 0.66-0.54 (m, 9H). ¹³C NMR (100 MHz, CD₃OD) δ 172.50, 136.59, 130.69, 130.45, 129.82, 129.69, 129.05, 128.67, 127.96, 65.64, 57.35, 55.55, 53.96, 50.89, 50.11, 49.70, 24.39, 18.60, 18.04, 10.95, 10.57, 8.71.

### Synthesis of (3S,6S,9S)-4,7-dibenzyl-3,6,9-triethyl-1,4,7-triazonan-2-one, 11

Compound 10 (15 g, 21 mmol) was dissolved in acetonitrile (2 L), added N-methylmorpholine (21 g, 0.21 mol), then added HATU (12 g, 32 mmol), after stirring at room temperature for 1 hour, the mixture was concentrated in vacuum, added water (500 mL), stirred for 2 hours, filtered to get the crude product, then the solid was recrystalized with methanol to get the product as a white solid (4.2 g, 49%), analytical HPLC showed the purity 95%. ¹H NMR (400 MHz, Acetonitrile-d₃) δ 7.43-6.13 (m, 10H), 4.02-2.09 (m, 11H), 1.74-0.77 (m, 6H), 0.77-0.26 (m, 9H). ¹³C NMR (100 MHz, CD₃CN) δ 174.08, 137.57, 130.94, 130.34, 130.00, 129.79, 129.66, 129.46, 129.43, 129.40, 129.15, 128.96, 128.10, 61.38, 57.61, 54.89, 52.54, 51.53, 50.76, 50.48, 24.68, 19.96, 18.20, 17.99, 14.67, 10.06, 9.72.

### Synthesis of (2S,5S,8S)-2,5,8-triethyl-1,4,7-triazonane, 13

Into a solution of compound 11 (3.0 g, 7.4 mmol) in dry THF (50 mL) was added 1 M LiAlH₄ in THF (15 mL, 15 mmol) slowly at room temperature, the mixture was refluxed for 2 days. Cooled down the temperature to 0-10° C., added ethyl acetate (3 mL), methanol (3 mL), then water (0.6 mL), then 15% NaOH solution (0.6 mL) and water (1.8 mL), filtered and the filter cake was washed with ethyl acetate (150 mL), the filtrate was concentrated in vacuum and then dissolved in trifluoroethanol (50 mL), added ammonium acetate (3 g) and Pd(OH)₂/C (loading 20 wt %, wet with 50% water) (1 g), then refluxed for 24 hours, cooled down the temperature and filtered, the filtrate was concentrated in vacuum, the residue was purified by revered-phase semi-preparative HPLC (mobile phase A: water with 1% TFA; mobile phase B: acetonitrile). This resulted in the product in the form of TFA salt (2.8 g, 75%). ¹H NMR (400 MHz, Methanol-d₄) δ 3.32-3.28 (m, 3H), 3.22-3.17 (m, 3H), 3.04-2.98 (m, 3H), 1.72-1.42 (m, 6H), 0.95 (t, J=7.5 Hz, 9H). ¹³C NMR (100 MHz, CDCl₃) δ 56.98, 55.17, 23.64, 9.35.

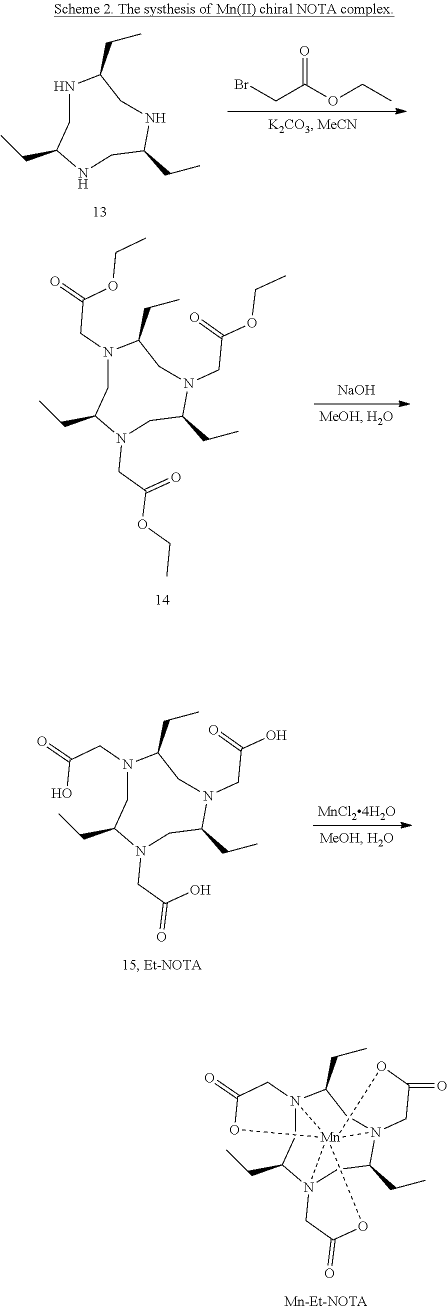

### Synthesis of 2,2′,2″-((2S,5S,8S)-2,5,8-triethyl-1,4,7-triazonane-1,4,7-triyl)triacetic acid, 15

Compound 13 (TFA salt) (1 g, 2.0 mmol) was dissolved in acetonitrile (15 mL), added K₂CO₃ (1.7 g, 12.3 mmol) and ethyl 2-bromoacetate (1.2 g, 7.2 mmol), after stirring at 50° C. for 16 hour, the reaction mixture was filtered and the filtrate was concentrated in vacuum, the resulted oil was dissolved in methanol (5 mL), added NaOH (0.35 g, 8.6 mmol) in water (3 mol), the reaction mixture was stirred at room temperature for 16 hours, concentrated in vacuum to remove most of the methanol, the residue was purified by reversed-phase semi-preparative HPLC (mobile phase A: water with 1% TFA; mobile phase B: acetonitrile). This resulted in the product in the form of TFA salt (0.8 g, 59%). ¹H NMR (400 MHz, D₂O) δ 3.92 (d, J=17.9 Hz, 3H), 3.74 (d, J=17.9 Hz, 3H), 3.30-3.14 (m, 3H), 2.93-2.71 (m, 6H), 1.68 (m, 3H), 1.24 (m, 3H), 0.80 (t, J=7.5 Hz, 9H). ¹³C NMR (100 MHz, D₂O) δ 60.75, 53.94, 47.43, 19.08, 9.98.

### Synthesis of Mn-Et-NOTA

Ligand 15 (TFA salt) (300 mg, 0.44 mmol) was dissolved in water (3 mL), methanol (3 mL), then added MnCl₂·4H₂O (130 mg, 0.66 mmol), the pH value of the mixture was adjusted to 6.85 by adding 0.1 M NaOH, after stirring at 70° C. for 16 hour, the reaction mixture was filtered and the filtrate was purified by reversed-phase semi-preparative HPLC (mobile phase A: 10 mM ammonium acetate; mobile phase B: 10 mM ammonium acetate/acetonitrile=1:9). The fraction of the product was dried through lyophilization, this resulted in the complex as a white powder (160 mg, 83%). m/z (ESI-MS⁺) 441.1673 ([M+2H]⁺ calculated: 441.1672).

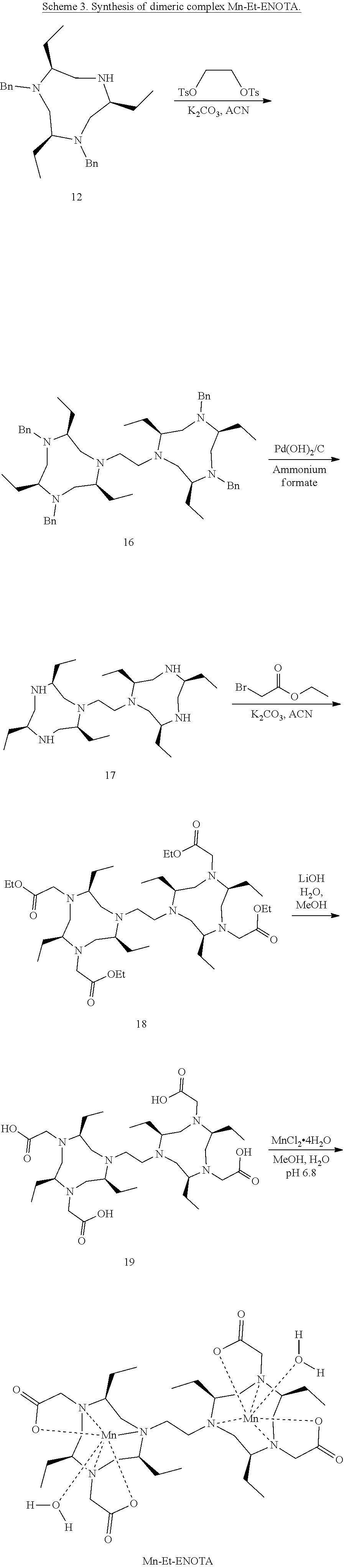

### Synthesis of 1,2-bis((2S,5S,8S)-2,5,8-triethyl-1,4,7-triazonan-1-yl)ethane, 17

Compound 12 (600 mg, 1.5 mmol) was dissolved in acetonitrile (10 mL), added K₂CO₃ (621 mg, 4.5 mmol) and ethane-1,2-diyl bis(4-methylbenzenesulfonate) (593 mg, 1.6 mmol), after stirring at 50° C. for 16 hour, the reaction mixture was filtered and the filtrate was concentrated in vacuum, the resulted oil was dissolved in trifluoroethanol (20 mL), added ammonium acetate (1.5 g) and Pd(OH)₂/C (loading 20 wt %, wet with 50% water) (0.5 g), then refluxed for 12 hours, cooled down the temperature and filtered, the filtrate was concentrated in vacuum, the residue was purified by revered-phase semi-preparative HPLC (mobile phase A: water with 1% TFA; mobile phase B: acetonitrile). This resulted in the product as a mixture of TFA and TsOH salts (0.8 g). ¹H NMR (400 MHz, Methanol-d₄) δ 3.62-3.60 (m, 2H), 3.35-3.32 (m, 2H), 3.24-2.93 (m, 6H), 2.89-2.51 (m, 10H), 2.30 (dd, J=15.7, 4.1 Hz, 2H), 1.66-1.17 (m, 10H), 1.09-1.06 (m, 2H), 0.89-0.37 (m, 18H). ¹³C NMR (100 MHz, CD₃OD) δ 59.71, 57.63, 56.42, 50.19, 45.42, 39.28, 37.01, 21.23, 20.86, 18.89, 9.88, 9.02, 8.99. m/z (ESI-MS⁺) 388.2449 ([M+H]⁺ calculated: 388.2499).

### Synthesis of 2,2′,2″,2′″-((2S,2′S,5S,5′S,8S,8′S)-ethane-1,2-diylbis(2,5,8-triethyl-1,4,7-triazonane-7,1,4-triyl))tetraacetic acid, 19

Compound 17 (TFA and TsOH salts) (800 mg) was dissolved in acetonitrile (15 mL), added K₂CO₃ (2.1 g, 15.2 mmol) and ethyl 2-bromoacetate (646 mg, 3.9 mmol), after stirring at 50° C. for 16 hour, the reaction mixture was filtered and the filtrate was concentrated in vacuum, the resulted oil was dissolved in methanol (4 mL), added NaOH (0.24 g, 6.0 mmol) in water (2 mol), the reaction mixture was stirred at room temperature for 16 hours, concentrated in vacuum to remove most of the methanol, the residuce was purified by reversed-phase semi-preparative HPLC (mobile phase A: water with 1% TFA; mobile phase B: acetonitrile). This resulted in the product in the form of TFA salt (0.8 g, 59%). ¹H NMR (400 MHz, Deuterium Oxide) δ 3.82 (d, J=17.7 Hz, 2H), 3.62 (d, J=18.0 Hz, 2H), 3.52 (d, J=17.7 Hz, 2H), 3.38 (d, J=17.9 Hz, 2H), 3.26 (s, 3H), 3.19-3.08 (m, 3H), 2.97-2.42 (m, 16H), 1.65-1.14 (m, 12H), 0.90-0.60 (m, 18H). ¹³C NMR (100 MHz, CD₃OD) δ 174.12, 172.07, 62.20, 61.49, 60.80, 54.96, 53.77, 50.29, 48.26, 46.50, 20.43, 19.77, 19.12, 10.44, 10.36, 10.12. m/z (ESI-MS⁺) 685.49 ([M+H]⁺ calculated: 685.41).

### Synthesis of Mn-Et-ENOTA

Ligand 19 (TFA salt) (160 mg, 0.13 mmol) was dissolved in water (2 mL), methanol (2 mL), then added MnCl₂·4H₂O (51 mg, 0.26 mmol), the pH value of the solution was adjusted to 6.8 by adding 0.1 M NaOH, after stirring at 70° C. for 16 hour, the reaction mixture was filtered and the filtrate was purified by reversed-phase semi-preparative HPLC (mobile phase A: 10 mM ammonium acetate; mobile phase B: 10 mM ammonim acetate/acetonitrile=1:9). The fraction of the product was dried through lyophilization, this resulted in the complex as a white powder (81 mg, 78%). m/z (ESI-MS⁺) 685.49 ([M+H]⁺ calculated: 685.41).

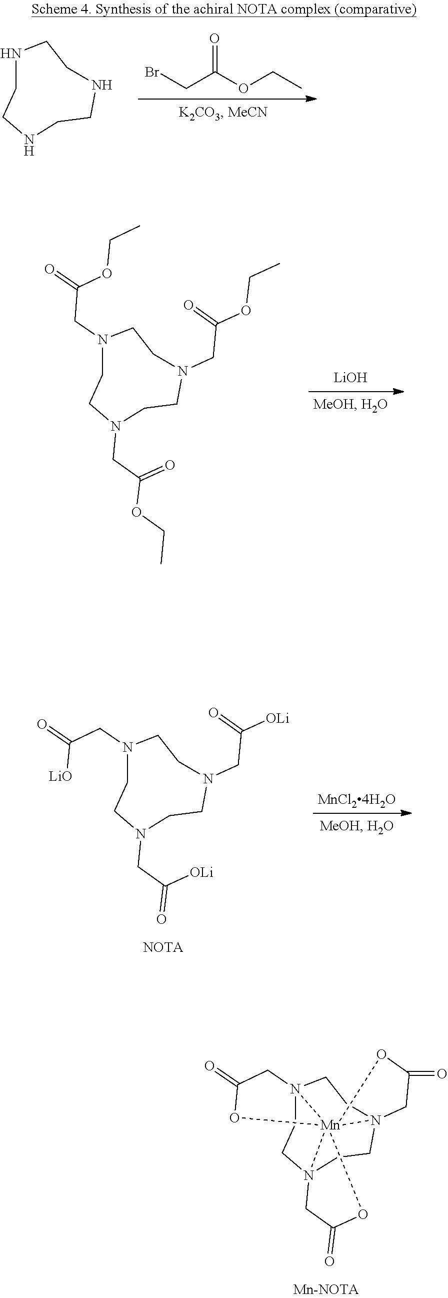

### Synthesis of 2,2′,2″-(1,4,7-triazonane-1,4,7-triyl)triacetic acid, NOTA

1,4,7-triazonane (2 g, 15.5 mmol) was dissolved in acetonitrile (25 mL), added K₂CO₃ (1.07 g, 77.5 mmol) and ethyl 2-bromoacetate (9.3 g, 55.8 mmol), after stirring at 50° C. for 16 hour, the reaction mixture was filtered and the filtrate was concentrated in vacuum, the resulted oil was dissolved in methanol (10 mL), added LiOH (1.6 g, 67.0 mmol) in water (5 mol), the reaction mixture was stirred at room temperature for 16 hours, concentrated in vacuum to remove most of the solvent, then added ethanol (3 mL), diethyl ether (15 mL), filtered and washed with diethyl ether, dried in vacuum and this resulted in the product in the form of lithium salt (3.2 g, 65%). ¹H NMR (400 MHz, Deuterium Oxide) δ 4.41 (s, 6H), 3.28 (s, 3H), 2.94 (s, 3H), 2.69 (s, 6H). ¹³C NMR (100 MHz, D₂O) δ 180.01, 170.36, 63.18, 59.48, 58.62, 57.76, 51.90.

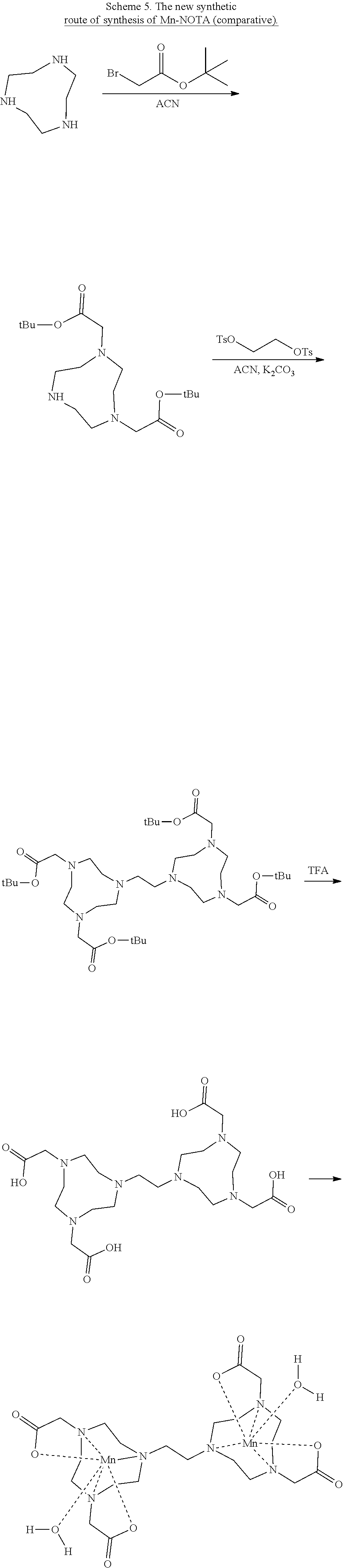

^{t}BuNOTA was synthesized according to the literature method.

### Synthesis of Tetra-tert-butyl 2,2′,2″,2′″-(ethane-1,2-diylbis(1,4,7-triazonane-7,1,4-triyl))tetraacetate

^{t}BuNOTA (400 mg, 1.1 mmol) was dissolved in acetonitrile (8 mL), then added K₂CO₃ (700 mg, 5.5 mmol) and ethane-1,2-diyl bis(4-methylbenzenesulfonate) (222 mg, 0.6 mmol), the reaction mixture was stirred at 50° C. for 16 hours, then the temperature was cooled down to room temperature, filtered and concentrated in vacuum, the crude product was purified by silica gel column chromatography which was eluted with dichloromethane/methanol (50:1-5:1). This resulted in the product as a foamy solid (612 mg, 74%), partial of the compound was in the form of TsOH salt as shown on NMR spectra. ¹H NMR (400 MHz, Chloroform-d) δ 3.30 (s, 8H), 3.00-2.51 (m, 28H), 1.44 (d, J=3.2 Hz, 36H). ¹³C NMR (100 MHz, CDCl₃) δ 171.49, 80.73, 77.22, 59.77, 55.95, 55.34, 53.41, 28.23.

### Synthesis of 2,2′,2″,2′″-(ethane-1,2-diylbis(1,4,7-triazonane-7,1,4-triyl))tetraacetic acid

Compound of tetra-tert-butyl 2,2′,2″,2′″-(ethane-1,2-diylbis(1,4,7-triazonane-7,1,4-triyl))tetraacetate (600 mg) was dissolved in dichloromethane (4 mL), then added TFA (4 mL), the mixture was stirred at room temperature for 16 hours, concentrated in vacuum, this resulted in the product as a light yellow oil (in the forms of TFA and TsOH salts) (900 mg). ¹H NMR (400 MHz, Deuterium Oxide) δ 3.91 (s, 8H), 3.46 (bs, 4H), 3.33-3.20 (m, 24H). ¹³C NMR (100 MHz, D₂O) δ 172.05, 56.69, 52.77, 50.32, 50.05, 49.77.

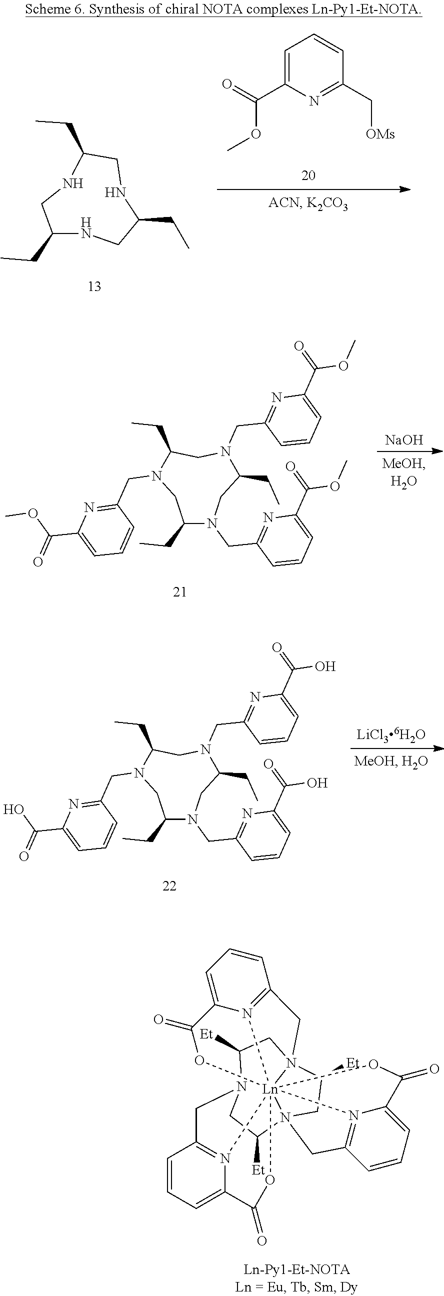

### Synthesis of 6,6′,6″-(((2S,5S,8S)-2,5,8-triethyl-1,4,7-triazonane-1,4,7-triyl)tris(methylene))tripicolinic acid, 22

Compound 13 (TFA salt) (80 mg, 0.16 mmol) was dissolved in acetonitrile (5 mL), added K₂CO₃ (218 mg, 16 mmol) and compound 20 (196 mg, 0.8 mmol), the mixture was stirred at 50° C. for 16 hours, the temperature was cooled down, filtered and the filtrate was concentrated in vacuum, then added methanol (5 mL) and NaOH (64 mg, 1.6 mmol) in water (2 mL), after stirring at 50° C. for 12 hours, then purified by revered-phase semi-preparative HPLC (mobile phase A: water with 1% TFA; mobile phase B: acetonitrile). This resulted in the product in the form of TFA salt. ¹H NMR (400 MHz, Methanol-d₄) δ 7.53 (t, J=7.8 Hz, 3H), 7.31 (d, J=7.9 Hz, 3H), 7.18 (d, J=7.8 Hz, 3H), 4.74 (s, 4H), 4.55 (s, 6H), 3.60 (bs, 3H), 3.43-3.40 (m, 3H), 2.14-1.90 (m, 3H), 1.73-1.44 (m, 3H), 0.96 (t, J=7.4 Hz, 9H). ¹³C NMR (100 MHz, CD₃OD) δ 165.99, 145.55, 138.66, 126.73, 124.62, 63.98, 57.90, 50.36, 18.92, 9.82.

### Synthesis of Ln-Py1-Et-NOTA

The general procedure of synthesis of Ln-Py1-Et-NOTA is as follows: the ligand 22 (TFA salt) (50 mg, 0.05 mmol) was dissolved in water (2 mL), then added LnCl₃·6H₂O (1.05 eq.), the pH value was adjusted to 7.0 and the mixture was stirred at 60° C. for 3 hours. The product was purified by purified by revered-phase semi-preparative HPLC (mobile phase A: water with 0.5% TFA; mobile phase B: acetonitrile). The fraction of the product was dried through lyophilization, this resulted in the complex as a white powder (yield˜85%).

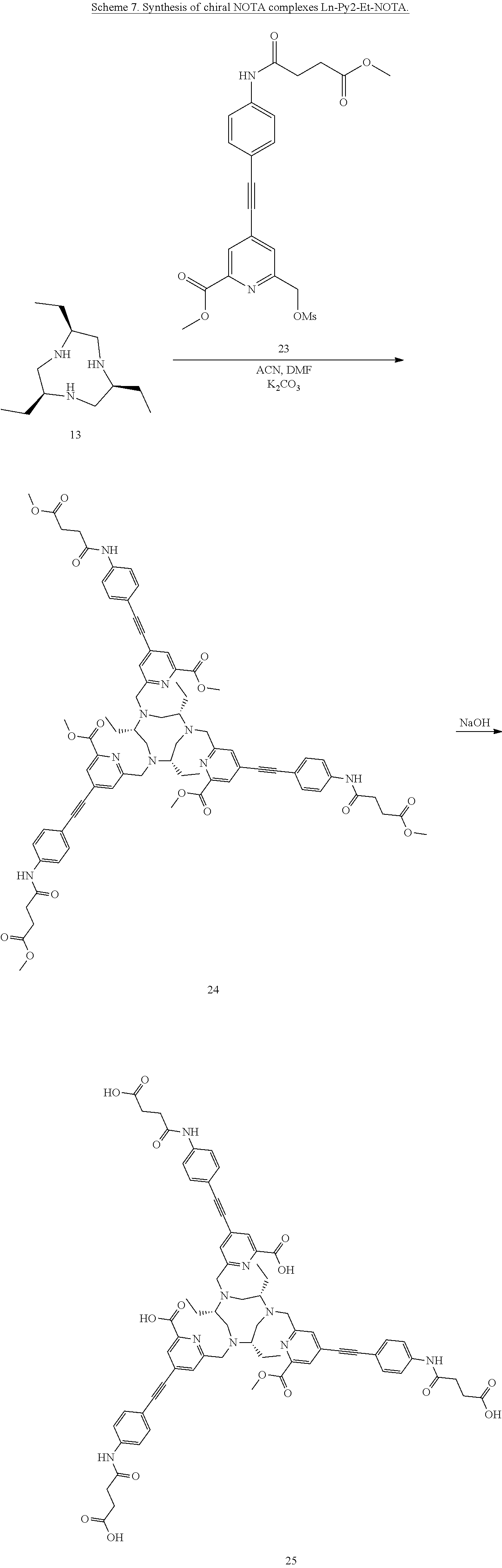 [SMILES: CC[C@H]1CN[C@@H](CC)CN[C@@H](CC)CN1 ; COC(=O)CCC(=O)Nc1ccc(C#Cc2cc(COS(C)(=O)=O)nc(C(=O)OC)c2)cc1 ; CC[C@H]1CN(Cc2cc(C#Cc3ccc(NC(=O)CCC(=O)OC)cc3)cc(C(=O)OC)n2)[C@@H](CC)CN(Cc2cc(C#Cc3ccc(NC(=O)CCC(=O)OC)cc3)cc(C(=O)OC)n2)[C@@H](CC)CN1Cc1cc(C#Cc2ccc(NC(=O)CCC(=O)OC)cc2)cc(C(=O)OC)n1 ; "NaOH" ; CC[C@H]1CN(Cc2cc(C#Cc3ccc(NC(=O)CCC(=O)O)cc3)cc(C(=O)O)n2)[C@@H](CC)CN(Cc2cc(C#Cc3ccc(NC(=O)CCC(=O)O)cc3)cc(C(=O)OC)n2)[C@@H](CC)CN1Cc1cc(C#Cc2ccc(NC(=O)CCC(=O)O)cc2)cc(C(=O)O)n1]

### Synthesis of Chiral NOTA Complexes Ln-Py2-Et-NOTA

Compound 13 (TFA salt) (50 mg, 0.1 mmol) was dissolved in acetonitrile (5 mL), added K₂CO₃ (138 mg, 1 mmol), the solution was heated to 55° C. and then added the solution of compound 23 (474 mg, 1 mmol) in DMF (4 mL) slowly (5 hours), the mixture was stirred at 55° C. for another 12 hours, the temperature was cooled down, filtered and concentrated to remove acetonitrile, then added water (30 mL), stirred for 30 mins and then filtered to get the crude product. It was dissolved in methanol (4 mL), added NaOH (24 mg, 0.6 mmol) in water (2 mL), the stirred at 60° C. for 20 mins, and then room temperature for overnight, the product was purified by revered-phase semi-preparative HPLC (mobile phase A: water with 1% TFA; mobile phase B: methanol). This resulted in the product in the form of TFA salt (15 mg). ¹H NMR (400 MHz, DMSO-d₆) δ 10.64 (s, 3H), 8.38-8.32 (m, 6H), 8.07 (d, J=8.3 Hz, 6H), 7.87 (d, J=8.3 Hz, 6H), 4.73-4.34 (m, 6H), 3.66-2.80 (m, 21H), 1.87-1.62 (m, 6H), 1.19 (t, J=7.3 Hz, 9H). ¹³C NMR (100 MHz, DMSO-d₆) δ 174.24, 171.01, 165.83, 157.27, 149.24, 141.17, 133.21, 128.64, 128.02, 119.16, 118.52, 115.20, 96.06, 85.82, 61.22, 57.96, 46.46, 31.61, 29.12, 20.00, 11.06.

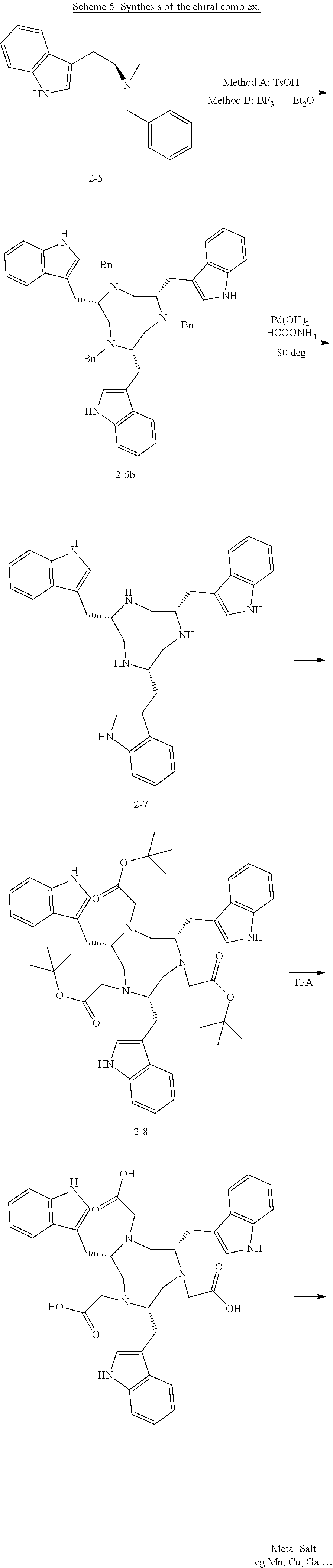

Synthesis of the chiral complex is prepared as outlined in Scheme 5.

What is claimed is:

1. A chiral NOTA chelator of Formula 1: 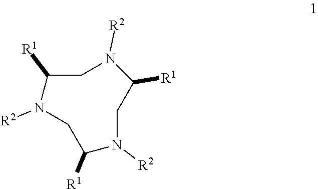
or a pharmaceutically acceptable salt or zwitterion thereof, wherein
R¹ is selected from the group consisting of hydrogen, alkyl, alkene, alkyne, cycloalkyl, heterocycloalkyl, aryl, heteroaryl, araalkyl, and —(CR₂)ₙY, wherein n is a whole number selected from 1-10; each R is independently hydrogen, alkyl, cycloalkyl, or aryl; or two R taken together with the carbon(s) to which they are attached form a 3-6 membered cycloalkyl; and Y is hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R¹ is a side chain of a naturally occurring amino acid or the side chain of a D-isomer of a naturally occurring amino acid; or R¹ is a moiety having the structure: 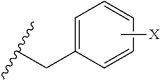
wherein X is azide, alkyne, halide, tosylate, mesylate, or hydroxyl; and
R² is —(C═O)OH, —(C═O)NHR⁵, or —(CH₂)ₘZ, wherein m is a whole number selected from 2-8; R⁵ is a targeting agent; and Z is moiety of Formula 2: 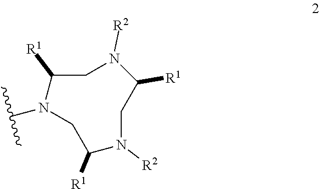
or a pharmaceutically acceptable salt or zwitterion thereof; or R² is a moiety of Formula 3: 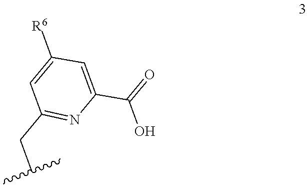
or a pharmaceutically acceptable salt or zwitterion thereof, wherein R⁶ for each occurrence is independently hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R⁶ is a moiety of Formula 4: 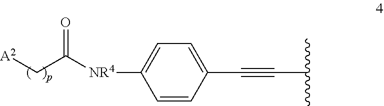
wherein p is a whole number selected from 1-6;
each A² is independently —CO₂R⁵, —NHR⁵, —OR⁵, N₃, or alkyne; and R⁴ is hydrogen or alkyl, with the proviso that if one R² is —(CH₂)₂Z and four R² are each —(C═O)OH, then each R¹ cannot be hydrogen; and if three R² are each —(C═O)OH, then each R¹ cannot be hydrogen.

2. The chiral NOTA chelator of claim 1, wherein each R¹ is selected from the group consisting of hydrogen, alkyl, aryl, heteroaryl, and —(CR₂)ₙY, wherein Y is heteroaryl or aryl; and n is 1-4.

3. The chiral NOTA chelator of claim 1 or 2, wherein each R² is —(C═O)OH; or each R² is —(C═O)NHR⁵.

4. The chiral NOTA chelator of claim 1, wherein the chiral NOTA chelator has Formula 5: 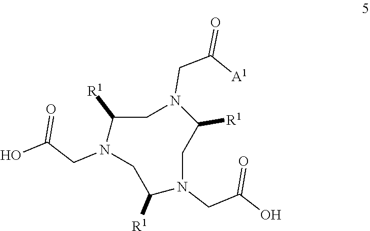
or a pharmaceutically acceptable salt or zwitterion thereof, wherein
A¹ is OH or NHR⁵;
each R¹ is selected from the group consisting of hydrogen, alkyl, aryl, heteroaryl, and —(CR₂)ₙY, wherein Y is heteroaryl or aryl; and n is 1-4; and
R⁵ is a targeting agent.

5. The chiral NOTA chelator of claim 4, wherein each R¹ is C₁-C₆ alkyl; or each R¹ is —(CR₂)ₙY, wherein n is a whole number selected from 1-4; and Y is aryl or heteroaryl.

6. The chiral NOTA chelator of claim 4, wherein each R¹ is ethyl; or each R¹ is 3-(λ³-methyl)-1H-indole.

7. The chiral NOTA chelator of claim 1, wherein the chiral NOTA chelator has Formula 7 or Formula 8 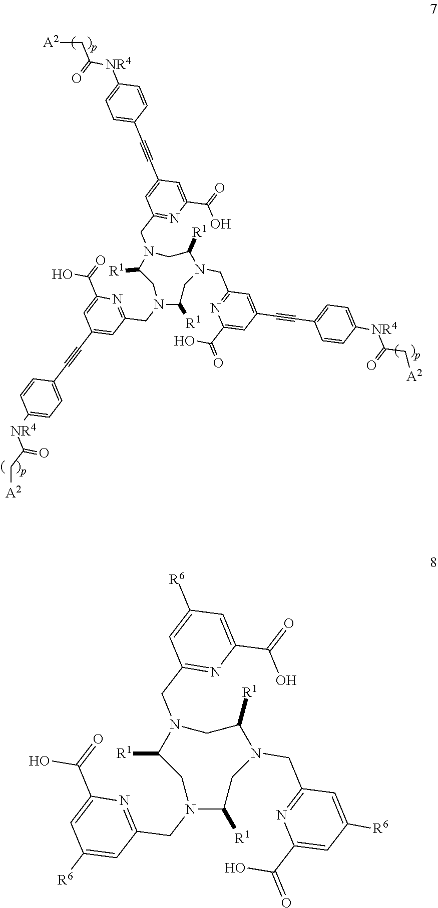
or a pharmaceutically acceptable salt or zwitterion thereof, wherein
p is a whole number selected from 1-4;
each A² is independently —CO₂R⁵, —NHR⁵, —OR⁵, N₃, or alkyne;
R¹ is selected from the group consisting of hydrogen, alkyl, aryl, heteroaryl, and —(CR₂)ₙY, wherein Y is heteroaryl or aryl; and n is 1-4;
R⁴ is hydrogen or alkyl;
R⁵ is hydrogen or a targeting agent; and
R⁶ for each occurrence is independently hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl.

8. The chiral NOTA chelator of claim 7, wherein each R¹ is C₁-C₆ alkyl; and R⁶ for each occurrence is independently hydrogen, alkyne, halide, —N₃, —R⁵, —NH₂, or —(C═O)OH.

9. The chiral NOTA chelator of claim 7, wherein p is a whole number selected from 1-2; each A² is independently —CO₂R⁵; each R¹ is C₁-C₆ alkyl; R⁴ is hydrogen; and R⁶ is hydrogen.

10. The chiral NOTA chelator of claim 9, wherein R¹ is ethyl; and R⁵ is hydrogen.

11. The chiral NOTA chelator of claim 1, wherein the chiral NOTA chelator has Formula 6: 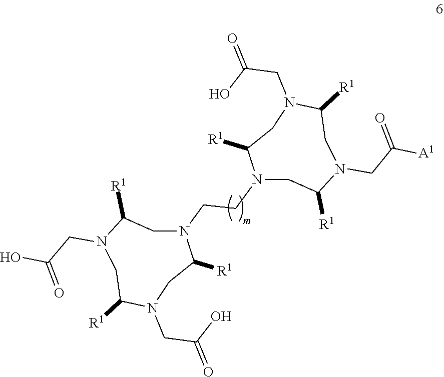
or a pharmaceutically acceptable salt or zwitterion thereof, wherein
m is a whole number selected from 2-8;
A¹ is OH or NHR⁵;
R¹ is selected from the group consisting of hydrogen, alkyl, alkene, alkyne, cycloalkyl, heterocycloalkyl, aryl, heteroaryl, araalkyl, and —(CR₂)ₙY, wherein n is a whole number selected from 1-10; each R is independently hydrogen, alkyl, cycloalkyl, or aryl; or two R² taken together with the carbon(s) to which they are attached form a 3-6 membered cycloalkyl; and Y is hydrogen, alkyl, alkene, alkyne, cycloalkyl, heterocycloalkyl, aryl, heteroaryl, cyano, halide, —N₃, —R⁵, —OR³, —OP(OR³)₃, —SR³, —NR³₂, —(C═O)OR³, —O(C═O)R³, —O(C═O)OR³, —(NR³)(C═O)R³, —(C═O)NR³₂, —O(C═O)NR³₂, —(NR³)(C═O)OR³, —(NR³)(C═O)NR³₂, or —(NR³)(C═NR³)NR³₂, wherein R³ for each instance is independently selected from hydrogen, alkyl, alkenyl, alkynyl, cycloalkyl heterocyloalkyl, aryl, and heteroaryl; or two R³ taken together with the atom(s) they are attached form a 3-7 membered cycloalkyl, 3-7 membered heterocycloalkyl, or 5 membered heteroaryl; or R¹ is a side chain of a naturally occurring amino acid or the side chain of a D-isomer of a naturally occurring amino acid; or R¹ is a moiety having the structure: 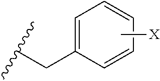
wherein X is azide, alkyne, halide, tosylate, mesylate, or hydroxyl; and
R⁵ is a targeting agent.

12. The chiral NOTA chelator of claim 11, wherein each R¹ is C₁-C₆ alkyl; and m is a whole number selected from 2-4.

13. The chiral NOTA chelator of claim 11, wherein R¹ is ethyl.

14. The chiral NOTA chelator of claim 1, wherein the chiral NOTA chelator is selected from the group consisting of: 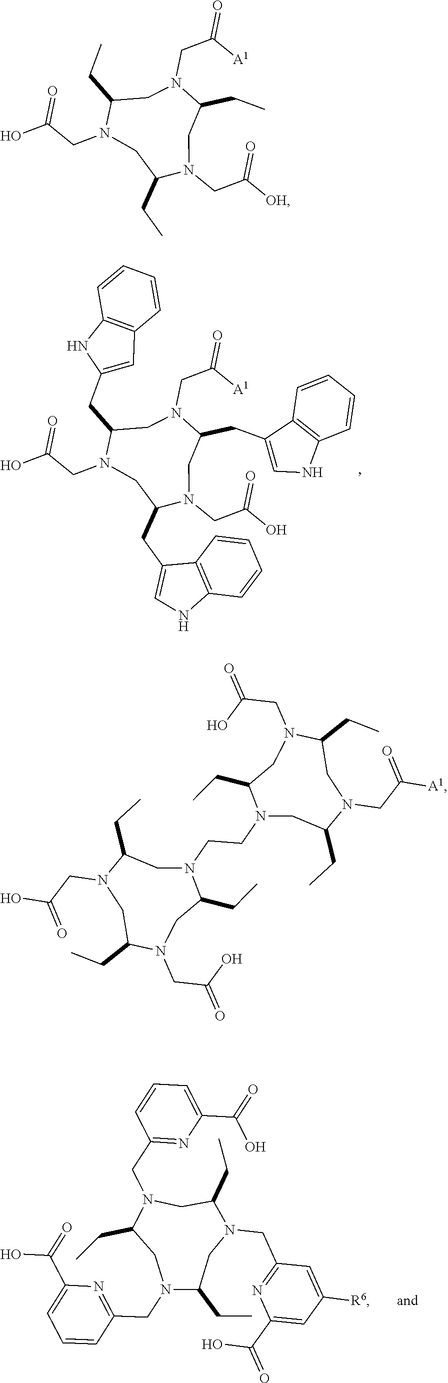 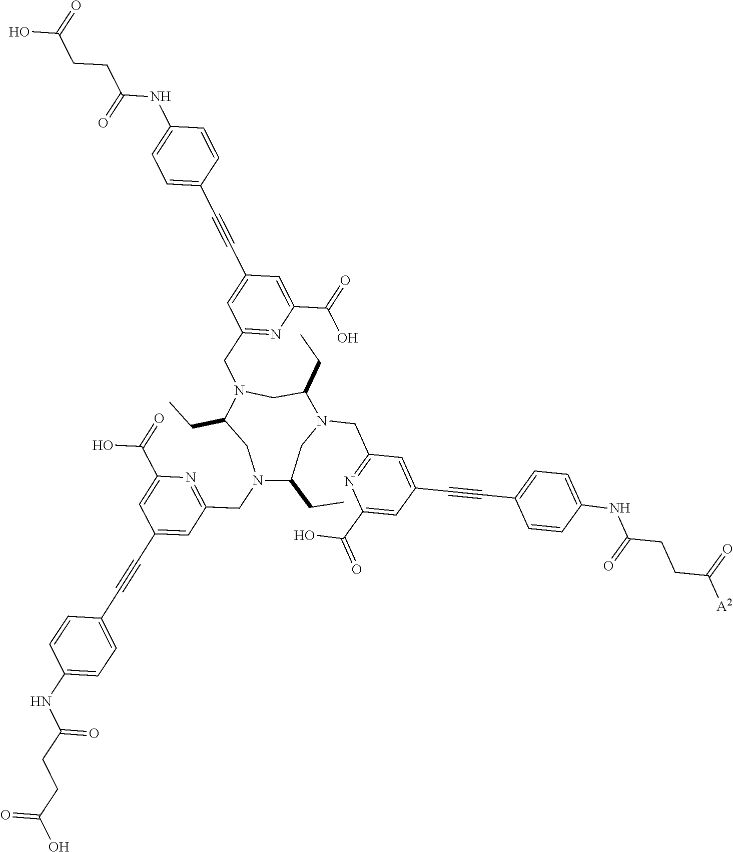
or a pharmaceutically acceptable salt or zwitterion thereof, wherein A¹ is OH or NHR⁵;
A² is OH or NHR⁵; and R⁶ is hydrogen or R⁵.

15. A chiral NOTA complex comprising the chiral NOTA chelator of claim 1 and at least one metal.

16. The chiral NOTA complex of claim 14, wherein the at least one metal is a Group 8-13 element of the periodic table, a lanthanide, or an actinide.

17. The chiral NOTA complex of claim 14, wherein the at least one metal is Gd, Eu, Tb, Lu, Yb, Y, In, or Mn.

18. A pharmaceutical composition comprising the chiral NOTA complex of claim 15 and at least one pharmaceutically acceptable excipient.

19. The chiral NOTA complex of claim 15 for use in imaging a sample.

20. The chiral NOTA complex for use of claim 19, wherein the imaging comprises positron emission tomography (PET), magnetic resonance imaging (MRI), computed tomography (CT) imaging, or optical imaging.

21. The chiral NOTA complex of claim 15 for use in imaging a subject.

22. The chiral NOTA complex for use of claim 20, wherein the imaging comprises positron PET, MRI, CT, or optical imaging.
